(19) United States
(12) Patent Application Publication
(10) Pub. No.: US 2026/0168860 A1
(43) Pub. Date: Jun. 18, 2026

(54) PASSIVE INFRARED SENSOR MOTION DETECTION SYSTEMS

(71) Applicant: XENEX DISINFECTION SERVICES INC., San Antonio, TX (US)

(72) Inventors: Nicholas Whitelonis, San Antonio, TX (US); Christopher Farquer, San Antonio, TX (US)

(21) Appl. No.: 19/416,168

(22) Filed: Dec. 11, 2025

Related U.S. Application Data
(60) Provisional application No. 63/733,238, filed on Dec. 12, 2024.

(51) Int. Cl. G01J 5/0806; A61L 2/084; A61L 2/102; A61L 2/104; A61L 2/24; A61L 103/75

(52) U.S. Cl. CPC G01J 5/0806; A61L 2/084; A61L 2/102; A61L 2/104; A61L 2/24; A61L 2103/75; A61L 2202/11; A61L 2202/14

(57) ABSTRACT

Motion detection systems are disclosed which include at least two PIR-based motion detection sensors configured such that when the sensing elements of their PIRs are oriented to detect horizontal movement, the motion sensors are vertically disposed. In some embodiments when the sensing elements are oriented in such a manner, the different optical systems of the motion sensors are configured such that a composite overlay of their horizontal fields of views has a beam pattern of one motion sensor overlapping a majority portion of dead zones of another sensor. In some cases, detection zones associated with one motion sensor have a higher density in a preset area than detection zones associated with another motion sensor and vice versa in a different preset area. Further to such systems, a disinfection apparatus is disclosed which includes at least two PIR-based motion detection sensors with different maximum detection distances.

### PRIORITY CLAIM

This application claims priority to U.S. Provisional Ser. No. 63/733,238 filed Dec. 12, 2024.

### BACKGROUND OF THE INVENTION

### 1. Field of the Invention

This invention generally relates to motion detection systems as well as apparatuses and systems incorporating such and, more particularly, to motion detection systems having at least two passive infrared motion detection sensors arranged such that their respective beam patterns increase detection sensitivity of the system.

### 2. Description of the Related Art

The following descriptions and examples are not admitted to be prior art by virtue of their inclusion within this section.

Passive infrared (PIR) based motion detection sensors are generally considered a low cost and low energy option for detecting motion. Their configuration and use vary, depending on the level of sensitivity desired and the areal coverage to be monitored. For example, a PIR-based motion detection sensor may be used to detect small movements in a confined area (such as monitoring movement of a door) and, thus, may be configured to have high sensitivity in a narrow range and, in some cases, may be placed relatively close to the area to monitored. In other cases, a PIR-based motion detection sensor may be used to detect large movements in wide area and/or a relatively far distance away (such as an individual walking across an area). In such cases, the sensitivity of a PIR-based motion detection sensor will generally not be as high since the sensitivity of PIR sensors decreases as distance from a sensor increases and also decreases with increasing detection angles.

PIR-based motion detection sensors are commonly used in security systems to detect the presence of an intruder. In addition, they are commonly used in home automation systems to monitor occupancy in a room or an area such that electronic devices (e.g., overhead lighting) in the room or area may be automatically controlled. In many long and/or wide area applications, the sensitivity of motion sensors in security systems and home automation systems need not be high since large movements are those that are desired to trigger an alarm or activate an electronic device in response thereto. In fact, in some security systems and home automation systems, PIR-based motion detection sensors are purposely configured to lessen their sensitivity to small movements, specifically to avoid or distinguish the movements of small animals (such as pets) from those of humans such that false intrusion alarms are not triggered, or electronic devices are not unnecessarily activated due to the presence of an animal.

Other systems known to utilize PIR-based motion detection sensors are automated room disinfection systems, particularly to terminate or inhibit operation of a system's germicidal source upon detecting motion indicative of a human or animal entering or moving in the room. In particular, automated room disinfection devices utilizing ultraviolet light, hydrogen peroxide vapor, and/or ozone to disinfect surfaces in a room often require tight monitoring of room occupancy to prevent inadvertent exposure to the germicide/s. In some cases, a room disinfection system may include a PIR-based motion detection sensor that is specifically configured and/or utilized to detect movement of a door to a room (i.e., the sensor may have a relatively narrow angle of detection and/or may be placed in alignment and, in some cases, in close proximity to the door). Although the use of such a motion detection sensor may have relatively high sensitivity to door movement, the sensor cannot be relied upon for detecting motion in other regions of the room. In view of this, a room disinfection system often additionally or alternatively includes a PIR-based motion detector sensor for monitoring motion in a wider region of the room and, in some embodiments, may include multiple PIR-based motion detection sensors to monitor an entire room.

Regardless of whether they are configured to detect door movement or motion in a wider region of a room, it is generally advantageous for the sensitivity of PIR-based motion sensor/s of a room disinfection system to be relatively high such that the room disinfection system's germicidal source can be reliably deactivated as quickly as possible to prevent harm to a person or animal entering the room. However, the use and/or configuration of room disinfection systems often present factors which counter such an objective for wide region monitoring, limiting the use of the PIR-based motion detection sensor/s to affect robust wide region motion detection. For example, it is generally advantageous for the areal coverage of PIR-based motion detection sensor/s of a room disinfection system to be relatively large to minimize the number of sensor/s in the system, but the increase in areal coverage comes at a cost of reduced sensitivity since the sensitivity of PIR-based motion detection sensors decrease with increasing detection angles.

In addition, although PIR-based motion detection sensor/s of a room disinfection system may be separate from the disinfection device in the system which includes the germicidal source, they are often arranged on the disinfection device to minimize the number of separate components making up the system. Although the latter arrangement is beneficial (e.g., avoids the risk of the motion detection sensor being misplaced from the room disinfection device and there are less items to set up for a disinfection cycle), the inherent nature of PIR-based motion detection sensors makes the arrangement less sensitive to movement unless the disinfection device is in close proximity to the area to be monitored. In particular, the sensitivity of a PIR sensor decreases with distance from the sensor due to the inverse square law. More specifically, the signal strength output by the PIR sensor is inversely proportional to the distance from the sensor. In addition, the detection zones produced by the optical system of the motion detection sensor for focusing infrared radiation onto the PIR sensor as well as the spaces between the detection zones become larger the greater the distance from the sensor. Larger detection zones and spaces therebetween reduce the sensitivity of detecting motion.

Furthermore, many room disinfection devices are portable such that they may be used in multiple rooms or areas of a facility. Such portability, however, induces a further hurdle in that the ability of the PIR-based motion detection sensor/s to reliably detect motion is dependent on the disinfection device being properly positioned such that the PIR sensor/s are directed toward and within a preset distance of an area to be monitored for movement. Moreover, a position of the disinfection device in a room to reliably detect motion may be different from a desired position of the disinfection device to sufficiently and/or efficiently disinfect highly targeted items in the room, such as but not limited to a bed in a patient room. Consequently, the objective of reliably detecting motion in a room may conflict with the objective to sufficiently and/or efficiently conduct a disinfection process in the room.

Yet another factor affecting motion detection via PIR-based motion detection sensors on a portable disinfection device is the variance of room configurations in which the device may be utilized. In particular, portable disinfection devices are often used in multiple rooms in a facility and the rooms are often of different sizes and dimensions and include different numbers and relative placement of egresses. Such room variances may induce efficacy disparities of PIR-based motion detection sensors among different rooms, which in turn may require a heightened level of awareness for properly positioning a portable disinfection device in rooms to achieve a particular level of efficacy in detecting motion is achieved. In particular, the proper placement and orientation of a room disinfection device in larger rooms or rooms with larger variance in dimensions will generally be more important in order to ensure the PIR-based motion detection sensors are arranged within a set distance and are directed to each area in the room to be monitored. Such an issue is also true and can be exacerbated when a room includes multiple doorways and monitoring motion at each of the doorways is desired.

Accordingly, it would be beneficial to develop PIR-based motion detection systems that have increased detection sensitivity, particularly for long and/or wide area applications. In addition, it would be advantageous to develop room disinfection systems including such, particularly having a PIR-based motion detection system which may effectively detect motion in a variety of room configurations.

### SUMMARY OF THE INVENTION

The following description of various embodiments of systems and apparatuses is not to be construed in any way as limiting the subject matter of the appended claims.

An embodiment of a motion detection system includes a first motion detection sensor having a first passive infrared sensor (PIR) and a first Fresnel lens array for focusing infrared radiation onto the first PIR. The motion detection system further includes a second motion detection sensor having a second PIR and a second Fresnel lens array for focusing infrared radiation onto the second PIR, wherein the second Fresnel lens array comprises a different configuration of lenses than the first Fresnel lens array. The first and second motion detection sensors are attached to and arranged on a substrate such that sensing elements of the first PIR and sensing elements of the second PIR can detect motion along the same plane in an ambient of the motion detection system. The motion detection system is such that when the sensing elements of the first and second PIRs are oriented to detect horizontal motion in the ambient of the motion detection system, the first motion detection sensor is arranged above and over the second motion detection sensor. In addition, when the sensing elements of the first and second PIRs are oriented in such a manner, the first and second Fresnel lens arrays are configured such that a composite overlay of their horizontal fields of views has a beam pattern associated with the first Fresnel lens array overlapping a majority portion of dead zones associated with the second Fresnel lens array. The motion detection system further includes detection circuitry independently and operatively coupled to the first PIR and the second PIR and which is configured to produce a motion detection signal upon receipt of a threshold signal from either the first PIR or the second PIR that indicates a predetermined change of infrared radiation has been detected at the respective PIR.

Another embodiment of a motion detection system includes a first motion detection sensor having a first PIR and a first optical system for focusing infrared radiation onto the first PIR, wherein the first optical system is configured to receive infrared radiation emanating from a first plurality of detection zones in an ambient of the first motion detection sensor. In addition, the motion detection sensor includes a second motion detection sensor having a second PIR and a second optical system for focusing infrared radiation onto the second PIR, wherein the second optical system is configured to receive infrared radiation emanating from a second plurality of detection zones in an ambient of the second motion detection sensor. One of the first and second motion detection sensors is vertically disposed above the other.

In addition, each of the first and second optical systems have a horizontal field of view embodying a first preset area extending from their respective central axis to a same first preset angle relative to their respective central axis and a second preset area extending from the same first preset angle to a same second preset angle relative to their respective central axis. The first plurality of detection zones associated with the first PIR includes a higher density of detection zones in its respective first preset area than the second plurality of detection zones associated with the second PIR includes in its respective first preset area. In addition, the second plurality of detection zones includes a higher density of detection zones in its respective second preset area than the first plurality of detection zones includes in its respective second preset area. The motion detection system further includes detection circuitry independently and operatively coupled to the first PIR and the second PIR and which is configured to produce a motion detection signal upon receipt of a threshold signal from either the first PIR or the second PIR that indicates a predetermined change of infrared radiation has been detected at the respective PIR.

Embodiments of apparatuses include either of the aforementioned motion detection systems. An embodiment of another apparatus includes a germicidal source and a motion detection system, wherein the apparatus is configured such that a germicide emitted from the germicidal source is projected exterior to the apparatus. The motion detection system of the apparatus includes a first motion detection sensor having a first PIR and a first optical system for focusing infrared radiation onto the first PIR and also includes a second motion detection sensor having a second PIR and a second optical system for focusing infrared radiation onto the second PIR. The first and second motion detection sensors include different maximum detection distances. The motion detection system of the apparatus further includes detection circuitry independently and operatively coupled to the first PIR and the second PIR. The detection circuitry is configured to send a motion detection signal to operational circuitry of the germicidal source upon receipt of a threshold signal from either the first PIR or the second PIR that indicates a predetermined change of infrared radiation has been detected at the respective PIR. In addition, the operational circuitry of the germicidal source is configured to affect operation of the germicidal source subsequent to receipt of the motion detection signal.

### BRIEF DESCRIPTION OF THE DRAWINGS

Other objects and advantages of the invention will become apparent upon reading the following detailed description and upon reference to the accompanying drawings in which:

FIG. 1 illustrates a front view of an example of a motion detection system having a pair of PIR-based motion detection sensors;

FIGS. 2A-2C illustrate top views of the motion detection system depicted in FIG. 1 respectively depicting different example arrangements of the pair of PIR-based motion detection sensors;

FIGS. 3A-3C illustrate side views of the motion detection system depicted in FIG. 1 respectively depicting different example arrangements of the pair of PIR-based motion detection sensors;

FIG. 4 illustrates a cross-sectional view of the motion detection system depicted in FIG. 3A taken along line A-A;

FIGS. 5-7 respectively illustrate a vertical field of view, a horizontal field of view, and a cross-sectional field of view of detection zones of the optical system of one of the PIR-based motion detection sensors of the motion detection system depicted in FIG. 1;

FIGS. 8-10 respectively illustrate a vertical field of view, a horizontal field of view, and a cross-sectional field of view of detection zones of the optical system of the other PIR-based motion detection sensor of the motion detection system depicted in FIG. 1;

FIG. 11 illustrates a composite overlay of the detection zones of the horizontal field of views of FIGS. 6 and 9;

FIG. 12 illustrates a composite overlay of the detection zones of the cross-sectional field of views of FIGS. 7 and 10;

FIG. 13 illustrates a schematic layout of redundant circuitry independently coupled between two PIR motion detection sensors and a system using motion detection signals generated by the circuitry;

FIGS. 14 and 15 respectively illustrate different example configurations of disinfection apparatuses including at least two PIR motion detection sensors; and

FIG. 16 illustrates an example top view of an apparatus having multiple PIR-based sensors arranged thereon to collectively monitor an area 360° around the apparatus for motion.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

The motion detection systems and apparatuses described herein incorporate at least two passive infrared (PIR) based motion detection sensors. Each of the PIR-based motion detection sensors includes a PIR sensor and an optical system for focusing infrared radiation on the respective PIR sensor. The PIR-based motion detection sensors are attached to and arranged on a substrate such that sensing element/s of the respective PIR sensors can detect motion along the same plane in an ambient of the motion detection system. The motion detection systems described herein further include detection circuitry independently and operatively coupled to the two PIR sensors. The detection circuitry is configured to produce a motion detection signal upon receipt of a threshold signal from either PIR sensor that indicates a predetermined change of infrared radiation has been detected at the respective PIR sensor.

As set forth in more detail below, the motion detection systems and apparatuses described herein may be of various configurations and may be used for various applications. For example, in some configurations, a motion detection system may be configured and arranged such that one of its PIR-based motion detection sensors is vertically disposed above another. In some such cases, the PIR-based motion detection sensors may be oriented such that when the PIR-based motion detection sensors are vertically disposed the central axes of their optical systems are substantially aligned with each other as viewed from a top view of the motion detection system. As explained in more detail below, the vertical arrangement of PIR-based motion detection sensors and an arrangement of sensing elements of their PIR sensors to detect motion along a horizontal plane enables a composite overlay of the horizontal field of views of the optical systems to represent a horizontal field of view of the motion detection system as a whole. In some embodiments, the PIR-based motion detection sensors may be additionally oriented such that the central axes of their optical systems are substantially parallel as viewed from a side view of the motion detection system. Furthermore, the PIR-based motion detection sensors may, in some embodiments, be arranged in close proximity to each other, such as being spaced less than approximately 10 cm from each other.

In any of such cases, the optical systems of the PIR-based motion detection sensors may have different configurations such that their respective beam patterns together increase detection sensitivity of the motion detection system. More specifically, the optical systems of the PIR-based motion detection sensors may be different and specifically configured to decrease the amount of dead zone area and/or increase the sensitivity in specific preset areas relative to their central axes. As a result, the detection sensitivity of the motion detection system as a whole may be higher as compared to using one or multiple PIR-based motion detection sensors with the same optical system configuration. In particular, one of the optical systems may have a beam pattern that overlaps a majority portion of dead zones of the other optical system/s as viewed with respect to their horizontal fields of views. In addition or alternatively, the beam patterns of the optical systems may have different densities of detection zones among various areas of their horizontal fields of views. Going forward in the disclosure herein, a system having at least two vertically disposed PIR-based motion detection sensors with optical systems having either or both of such different configurations may be referenced herein as a “PIR-based motion detection multiple sensor coalesced beam pattern system”. In general, the motion detection systems considered herein may include any number of PIR-based motion detection multiple sensor coalesced beam pattern systems, including a single system or multiple systems.

In other cases, a pair or more of PIR-based motion detection sensors in a motion detection system considered herein need not be configured to have coalesced beam patterns. Furthermore, a pair or more of PIR-based motion detection sensors in a motion detection system considered herein need not be arranged over one another, oriented such that the central axes of their optical systems are substantially aligned, and/or have different configurations of optical systems. In particular, a motion detection system having at least two PIR-based motion detection sensors with different detection ranges is also disclosed herein, in which the PIR-based motion detection sensors may or may not be vertically disposed, may or may not have the central axes of their optical systems substantially aligned with each other, and may or may not have optical systems with different configurations. The term “detection range” is referred to herein as the maximum distance which a PIR-based motion detection sensor may reliably detect motion. As such, an alternative manner to describe some of the motion detection systems considered herein is to say the motion detection systems include at least two PIR-based motion detection sensors having different maximum detection distances.

Regardless of the relative configuration of the at least two PIR-based motion detection sensors in the motion detection systems disclosed herein, the detection angles of their optical systems may be substantially similar or may be different. The term “detection angle” is referred to herein as the angle which a PIR-based motion detection sensor or a motion detection system as a whole may reliably detect motion with respect to a field of view along a particular axis (i.e., horizontal or vertical axis). The term “field of view” is referred to herein as the ambient area which a PIR-based motion detection sensor or system may reliably detect motion. The ambient area is a three-dimensional space but is often referenced by the two-dimensional space it occupies along a particular axis (i.e., a “horizontal field of view” or a “vertical field of view”). The width of a field of view along a particular axis is governed by the detection angle of the PIR-based motion detection sensor along that axis and may be the same or different from the detection angle of the PIR-based motion detection sensor along the opposing axis (i.e., the detection angle of a horizontal field of view may be the same or different than the detection angle of a vertical field of view).

In general, the detection angles of the optical systems of the PIR-based motion detection sensors discussed herein may generally be between approximately 15° and up to 180°, depending on the design specifications of the motion detection system. In addition, the detection ranges of the PIR-based motion detection sensors may generally be between approximately 0.5 feet and approximately 30 feet, depending on the design specifications of the motion detection system. As such, the motion detection systems described herein are not limited to the depictions in the drawings. Furthermore, although the motion detection systems disclosed herein are emphasized for use in conjunction with area/room disinfection apparatuses, the use of the motion detection systems need not be so limited. In particular, the motion detection systems considered herein may be used in conjunction with other systems, such as but not limited to security systems, home automation systems, and room occupancy monitoring systems that are used to prevent exposure to hazardous conditions, such as but not limited to systems for rooms containing X-ray computed tomography equipment.

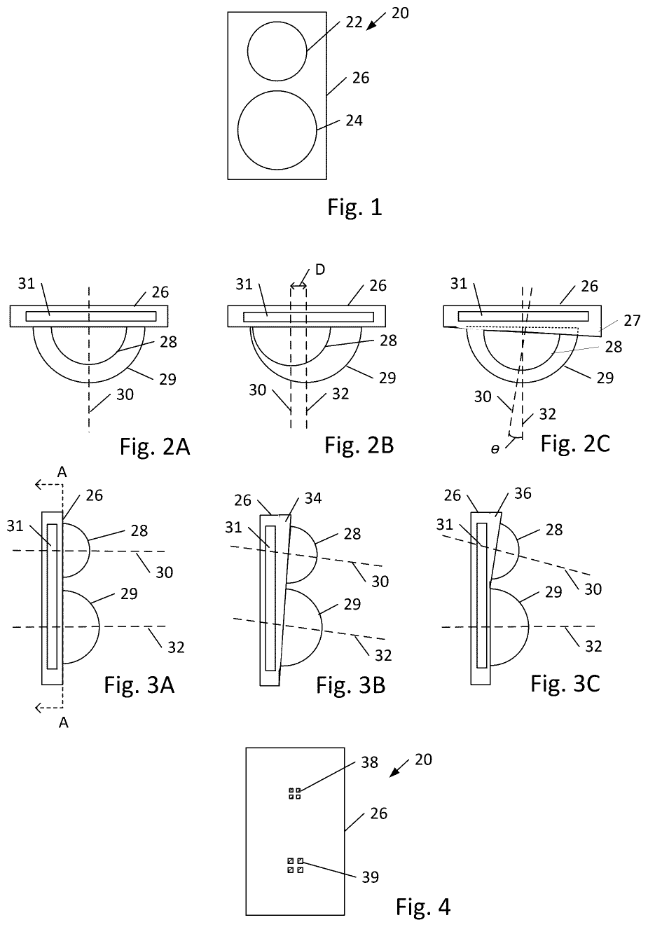

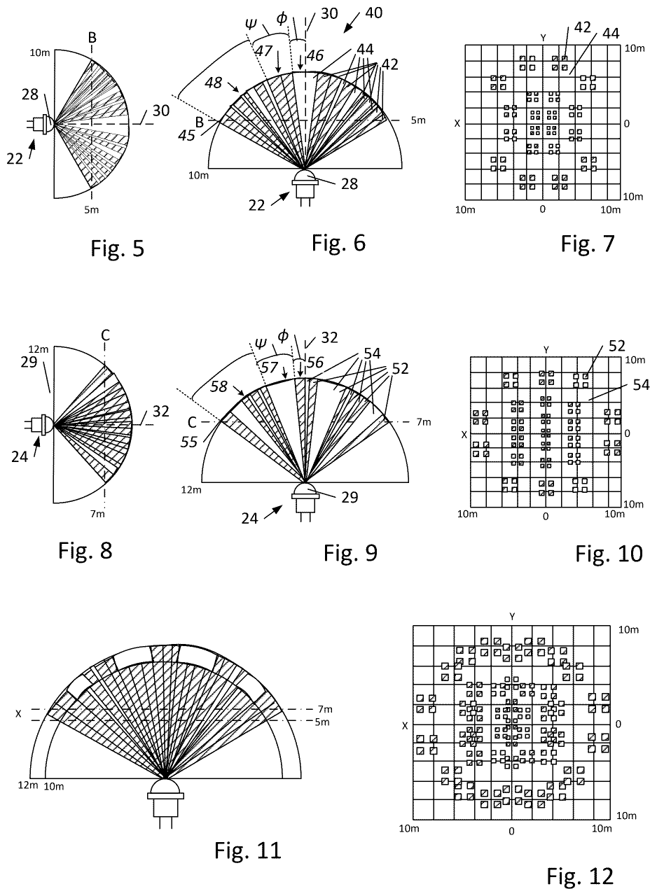

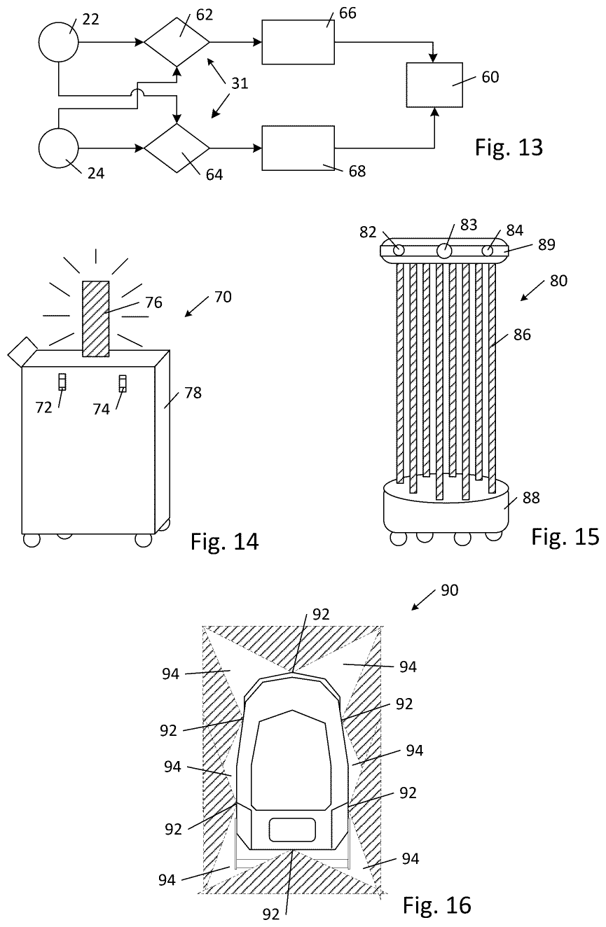

Turning to the drawings, FIGS. 1, 2A-2C, 3A-3C, and 4 illustrate example configurations of a motion detection system having a pair of PIR-based motion detection sensors arranged one over the other, in close proximity to each other, and such that their central axes are substantially aligned. In addition, FIGS. 5-12 illustrate examples of individual and composite overlays of the fields of views of the optical systems of the PIR-based motion detection sensors of FIGS. 1, 2A and 3A. FIG. 13 illustrates a schematic layout of example circuitry which may be included in any of the motion detection systems disclosed herein, specifically circuitry which is independently and operatively coupled between two PIR motion detection sensors and a system using motion detection signals generated by the circuitry. FIGS. 14 and 15 respectively illustrate different example configurations of area/room disinfection apparatuses having a germicidal source and at least two PIR motion detection sensors. FIG. 16 illustrates an example top view of an apparatus having multiple PIR sensors arranged thereon to collectively monitor an area 360° around the apparatus for motion.

As shown in FIG. 1, motion detection system 20 includes PIR-based motion detection sensor 22 arranged above and over PIR-based motion detection sensor 24 on substrate 26. In other cases, PIR-based motion detection sensor 24 may be arranged above and over PIR-based motion detection sensor 22 on substrate 26. In either configuration, PIR-based motion detection sensors 22 and 24 may be described as being vertically disposed along substrate 26. The phrase “above and over” and the term “vertically disposed” as used herein refers to the relative arrangement of two features in which at least a portion of the each of the two features overlap as viewed along and above a horizontal plane containing the features. Examples of PIR-based motion detection sensor 22 being arranged above and over PIR-based motion detection sensor 24 are shown in FIGS. 2A-2C and described in more detail below. As further described in reference to FIGS. 2A-2C, PIR-based motion detection sensors 22 and 24 may be arranged along substrate 26 and relative to each other such that the central axes of their optical systems are substantially aligned as viewed from a top view of motion detection system 20 when the pair of motion detection sensors are disposed in a vertical arrangement.

Although motion detection system 20 is shown in FIGS. 1-3C and described in more detail below as having a pair of PIR-based motion detection sensors, it is noted the PIR-based motion detection multiple sensor coalesced beam pattern systems disclosed herein are not necessarily restricted to having two PIR-based motion detection sensors. Rather, the PIR-based motion detection multiple sensor coalesced beam pattern systems disclosed herein may include any multiple of vertically disposed PIR-based motion detection sensors which are arranged such that the central axes of their optical systems are substantially aligned. Furthermore, similar to the description below for motion detection system 20, the beam patterns of the optical systems associated with a PIR-based motion detection multiple sensor coalesced beam pattern system having more than two PIR-based motion detection sensors may be collectively configured to increase detection sensitivity of the system. In particular, the beam patterns may have detection regions overlapping or underlying dead zones of another and/or have a variation of detection region densities relative to each other as is described in reference to FIGS. 5-12 for the pair of PIR-based motion detection sensors of motion detection system 20.

In some embodiments, substrate 26 may be a fixed component to which PIR-based motion detection sensors 22 and 24 are attached (i.e., substrate 26 is not moveable and, thus, the orientation of substrate 26 is not changeable). In such cases, PIR-based motion detection sensors 22 and 24 may be described as being vertically disposed on substrate 26. In other embodiments, substrate 26 may be a freestanding component and/or may be configured for attachment to another substrate. For example, substrate 26 (and thus motion detection system 20 as a whole) may be mounted to a vertical surface of a room in some embodiments. In other embodiments, substrate 26 (and thus motion detection system 20 as a whole) may be mounted along a sidewall of an apparatus. In such cases, PIR-based motion detection sensors 22 and 24 may be described as being arranged side-by-side along substrate 26 and substrate 26 may be oriented such that PIR-based motion detection sensors 22 and 24 are vertically disposed. In any case, PIR-based motion detection sensors 22 and 24 may, in some configurations, be coupled to the same printed circuit board within substrate 26, particularly if PIR-based motion detection sensors 22 and 24 are arranged in close proximity to each other, such as less than 10 cm from each other. In other embodiments, PIR-based motion detection sensors 22 and 24 may be coupled to different printed circuit boards within substrate 26.

In any case, PIR-based motion detection sensors 22 and 24 are shown as different sizes in FIG. 1 to indicate that they have different detection ranges. As noted above, the term “detection range” is referred to herein as the maximum distance which a PIR-based motion detection sensor may reliably detect motion. Although motion detection system 20 is shown having smaller PIR-based motion detection sensor 22 above larger PIR-based motion detection sensor 24 (i.e., a PIR-based motion detection sensor having a shorter detection range above a PIR-based motion sensor having a longer detection range) when the motion detection sensors are vertically disposed, their arrangement may be switched. As described in more detail below in reference to FIG. 11, a variance of detection ranges among PIR-based motion detection sensors 22 and 24 may allow motion to be detected at a relatively far distance in a given area from motion detection system 20 while also having relatively high sensitivity of detecting motion in a closer region in the same given area. Despite such an advantage, motion detection system 20 may alternatively have a pair of PIR-based motion detection sensors with substantially similar detection ranges. Such a configuration may be advantageous for having relatively consistent sensitivity across the detection angle of motion detection system 20.

PIR-based motion detection sensors 22 and 24 each include a PIR sensor and an optical system for focusing infrared radiation on their respective PIR sensor. The optical systems may include Fresnel lens arrays 28 and 29 such as shown in the top view and side view of motion detection system 20 in FIGS. 2A-2C and 3A-3C and/or may include segmented mirrors. In any case, the optical systems of PIR-based motion detection sensors 22 and 24 are different, particularly in their configurations to receive infrared radiation emanating from different distributions of detection zones in the ambient of motion detection system 20. Alternatively stated, the optical systems of PIR-based motion detection sensors 22 and 24 are different with respect to the distribution of detection zones from which they are configured to focus infrared radiation on to the respective PIR sensors of motion detection sensors 22 and 24. The variation among the optical systems may generally include a variation in the configuration (i.e., number, size and/or placement) of Fresnel lenses on Fresnel lens arrays 28 and 29 and/or a variation in the configuration (i.e., number, size and/or placement) of segmented mirrors comprising the optical systems.

Regardless of the configuration of their optical systems, PIR-based motion detection sensors 22 and 24 may be arranged along substrate 26 and relative to each other such that the central axes of Fresnel lens arrays 28 and 29 are substantially aligned with each other as viewed from a top view of motion detection system 20 shown in FIG. 1 (i.e., when the pair of PIR-based motion detection sensors are disposed in a vertical arrangement). The term “substantially aligned” as used herein refers to the relative arrangement of two lines which vary from each other by less than 1 cm and/or less than 5°. The term encompasses two parallel lines which are separated by less than 1 cm as well as two lines angled less than 5° relative to each other. As set forth in more detail below, having a pair of PIR-based motion detection sensors arranged such that the central axes of their optical systems are substantially aligned with each other as viewed from a top view of the motion detection system aids in the system being configured to have high detection sensitivity relative to what is offered by conventional systems with a similar detection angle. However, it is noted that the motion detection systems considered herein need not have the central axes of optical systems of a pair of PIR-based motion detection sensors substantially aligned to have beam patterns which overlap dead zones of the other or with different densities as discussed in more detail below in reference to FIGS. 5-12

The alignment design among optical systems was first developed having the pair of PIR-based motion detection sensors centered over one another such that the central axes of their optical systems overlap in their entirety as described in more detail below in reference to FIG. 2A. However, such preciseness is not necessarily needed to achieve a high level of detection sensitivity. Thus, the concept of having a pair of PIR-based motion detection sensors arranged such that the central axes of their optical systems are substantially aligned as defined above is described herein. The slight variation of alignment encompassed by the term “substantially aligned” is generally advantageous for tolerating variations in manufacturing and mounting the PIR-based motion detection sensors on a shared substrate.

The term “central axis” as used herein refers to a reference line passing through a center of an object, apparatus, or system and, along a given plane, symmetrically divides the object, apparatus, or system into equal halves. For instance, a central axis of a Fresnel lens array that is in the shape of a hollow spherical dome refers to a reference line passing through the geometric center and the apex of the spherical dome. It is noted that the central axis of an optical system, such as a Fresnel lens array, may or may not be the optical axis of the optical system. In particular, the term “optical axis” as used herein refers to a center line of a field of view of an optical system. Although many optical systems have fields of view centered along their central axis, some optical systems may have fields of view offset from their central axis and, thus, the central axis and the optical axis of an optical system may not be the same.

FIGS. 2A-2C illustrate different example orientations of PIR-based motion detection sensors 22 and 24 which constitute central axes 30 and 32 of their respective Fresnel lens arrays to be substantially aligned with each other as viewed from a top view of motion detection system 20. In particular, FIG. 2A illustrates a top view of motion detection system 20 depicting PIR-based motion detection sensors 22 and 24 configured such that central axis 30 overlaps central axis 32 (i.e., such that central axis 30 is arranged over and above central axis 32 in its entirety). Central axis 32 of Fresnel lens array 29 is not shown in FIG. 2A since central axis 30 overlaps it as whole. In order to facilitate such an overlap of central axis 30 over central axis 32, PIR-based motion detection sensor 22 is centered over PIR-based motion detection sensor 24 (i.e., the center point of Fresnel lens array 28 is arranged directly above and over the center point of Fresnel lens array 29) and PIR-based motion detection sensors 22 and 24 are oriented such that central axes 30 and 32 extend at the same angle from the center points of their respective Fresnel lens arrays with respect to the vertical plane of motion detection system 20.

FIG. 2B illustrates a different orientation of PIR-based motion detection sensor 22 relative to PIR-based motion detection sensor 24 which constitutes central axes 30 and 32 of their respective Fresnel lens arrays to be substantially aligned with each other as viewed from a top view of motion detection system 20. In particular, FIG. 2B illustrates a top view of motion detection system 20 depicting PIR-based motion detection sensors 22 and 24 arranged slightly vertically off-center from each other such that central axis 30 is spaced apart from central axis 32 by distance D. In accordance with definition of the term “substantially aligned” as used herein and specified above, distance D is less than 1 cm such that central axes 30 and 32 may be considered substantially aligned with each other. It is noted that for the alternative arrangement depicted in FIG. 2B, PIR-based motion detection sensors 22 and 24 would be arranged slightly off-center from each other in the viewpoint of FIG. 1 as well.

As shown in FIGS. 2A and 2B, central axes 30 and 32 may, in some embodiments, be parallel as viewed from a top view of motion detection system 20. The motion detection systems described herein, however, are not necessarily so limited. In particular, PIR-based motion detection sensors 22 and 24 may be configured such that central axis 30 is not parallel but yet substantially aligned with central axis 32, particularly by the two axes being angled less than 5° relative to each other as viewed from a top view of the motion detection system. Configurations of PIR-based motion detection sensors 22 and/or 24 to do so may include the configuration of their respective optical systems themselves and/or the angle at which PIR-based motion detection sensors 22 and/or 24 are mounted to substrate 26. FIG. 2C illustrates example orientations of PIR-based motion detection sensors 22 and 24 which constitute central axes 30 and 32 as non-parallel but angled less than 5° relative to each other as viewed from a top view of the motion detection system and, thus, central axes 30 and 32 are considered to be substantially aligned with each other in such a configuration.

In particular, FIG. 2C illustrates a top view of motion detection system 20 depicting PIR-based motion detection sensor 22 centered over PIR-based motion detection sensor 24, but PIR-based motion detection sensors 22 and 24 are mounted on an alternative configuration of substrate 26. In particular, PIR-based motion detection sensor 22 is mounted along angled addendum portion 27 of substrate 26 to angle it relative to PIR-based motion sensor 24. As shown by the dotted lines in substrate 26 in FIG. 2C, PIR-based motion detection sensor 24 is arranged along a rectangular base portion of substrate 26 which does not include addendum portion 27. As a consequence, central axis 30 is angled from central axis 32 by angle θ in FIG. 2C. In accordance with definition of the term “substantially aligned” as used herein and specified above, angle θ is less than 5° such that central axes 30 and 32 may be considered substantially aligned with each other.

Regardless of the configuration of PIR-based motion detection sensors 22 and 24 to have central axes 30 and 32 substantially aligned as viewed from a top view of motion detection system 20 when the PIR-based motion detection sensors are vertically disposed, the relative orientations of the PIR-based motion detection sensors may be such that central axes 30 and 32 are parallel, converge, or diverge as viewed from a side view of motion detection system 20 when the motion detection sensors are vertically disposed. Example arrangements of PIR-based motion detection sensors 22 and 24 to affect central axes 30 and 32 to be parallel and convergent are depicted in FIGS. 3A-3C and described in more detail below.

In general, the orientations of PIR-based motion detection sensors 22 and 24 to affect central axes 30 and 32 to be parallel, to converge, or to diverge as viewed from a side view of motion detection system 20 when the motion detection sensors are vertically disposed will affect the vertical coverage of the area to be monitored. In view thereof, different objectives for motion detection system 20 may warrant the central axes to be parallel, to converge, or to diverge from a side view of the motion detection system. For instance, in cases in which detecting motion from varying distances from the motion detection system is desired, it may be advantageous to orient PIR-based motion detection sensors 22 and 24 such that central axes 30 and 32 are parallel to ensure substantially similar vertical coverage of motion detection can be achieved at each of the varying distances. However, orienting PIR-based motion detection sensors 22 and 24 such that central axes 30 and 32 converge or diverge may also be considered for such scenarios.

In cases in which detecting motion at a certain distance from the motion detection system is desired, it may be advantageous to orient PIR-based motion detection sensors 22 and 24 such that central axes 30 and 32 converge to that approximate distance but orienting them such that central axes 30 and 32 diverge or are parallel may also be considered for such scenarios. In cases in which it may be advantageous to detect motion in an area having a larger vertical dimension than what may be offered by the detection angles of either PIR-based motion detection sensors 22 and 24 in their vertical fields of view, it may be advantageous to orient PIR-based motion detection sensors 22 and 24 such that central axes 30 and 32 diverge. In any case, it is noted that the orientations of PIR-based motion detection sensors 22 and 24 to affect central axes 30 and 32 to be parallel, to converge, or to diverge need not be dependent on the configuration of the PIR-based motion detection sensors to have beam patterns which overlap dead zones of the other or with different densities as discussed in more detail below in reference to FIGS. 5-12.

FIGS. 3A and 3B illustrate different example orientations of PIR-based motion detection sensors 22 and 24 in which central axes 30 and 32 are parallel as viewed from a side view of motion detection system 20. In particular, FIG. 3A illustrates a side view of motion detection system 20 depicting PIR-based motion detection sensors 22 and 24 mounted on a rectangular base portion of substrate 26 such that central axes 30 and 32 are parallel and extend in a horizontal direction from substrate 26. FIG. 3B, on the other hand, illustrates a side view of motion detection system 20 depicting PIR-based motion detection sensors 22 and 24 each mounted on an alternative configuration of substrate 26, particularly along angled addendum portion 34 of substrate 26 such that central axes 30 and 32 are parallel and extend in a downward direction from substrate 26. In a different configuration, PIR-based motion detection sensors 22 and 24 may each be mounted on an yet another alternative configuration of substrate 26, particularly along an addendum portion of substrate 26 which facilitates central axes 30 and 32 to be parallel and extend in an upward direction from substrate 26.

FIG. 3C illustrates different example orientations of PIR-based motion detection sensors 22 and 24 in which central axes 30 and 32 converge as viewed from a side view of motion detection system 20. In particular, FIG. 3C illustrates a side view of motion detection system 20 depicting PIR-based motion detection sensor 22 mounted on an alternative configuration of substrate 26, particularly along on angled addendum portion 36 of substrate 26. In addition, FIG. 3C illustrates PIR-based motion detection sensor 24 mounted a rectangular base portion of substrate 26 such that central axes 30 and 32 converge relative to each other. In an alternative configuration, PIR-based motion detection sensors 22 and 24 may be respectively mounted on opposing addendum portions of substrate 26 which facilitate central axes 30 and 32 to converge to a greater degree than the configuration depicted in FIG. 3B. In yet other configurations, PIR-based motion detection sensors 22 and 24 may be mounted on portions of substrate 26 (i.e., base portions or addendum portions or substrate 26) which facilitate central axes 30 and 32 to diverge relative to each other.

As noted above, in addition to their optical systems, PIR-based motion detection sensors 22 and 24 each include a PIR sensor. FIG. 4 illustrates a cross-sectional view of motion detection system 20 taken along line A-A of FIG. 3A illustrating sensing elements 38 and 39 of the respective PIR sensors of PIR-based motion detection sensors 22 and 24, which reside beneath Fresnel lens arrays 28 and 29. Although the PIR sensors of PIR-based motion detection sensors 22 and 24 are shown each having a quad set of sensing elements, their PIR sensors are not necessarily so limited. In particular, the PIR sensor of each of PIR-based motion detection sensors 22 and 24 may alternatively include a single sensing element or dual sensing elements. In any case, PIR-based motion detection sensors 22 and 24 are arranged on substrate 26 such that their sensing elements can detect motion along the same plane in an ambient of motion detection system 20. More specifically, the sensing element/s of PIR-based motion detection sensor 22 are oriented at the same angle as the sensing element/s of PIR-based motion detection sensor 24 such that the lengthwise and widthwise dimensions of their opposing sets of sensing elements are parallel.

As described in more detail below, the motion detection systems described herein may be specifically configured for detecting horizontal movement in an ambient of the systems. In order to affect such an objective, the pair of PIR-based motion detection sensors of a motion detection system may be arranged on a substrate (or a substrate having the motion detection sensors arranged thereon may be arranged) such that the sensing elements of the motion detection sensors are oriented to detect horizontal motion in the ambient of the system. For example, in a configuration in which a dual set of sensing elements are included in each PIR-based motion detection sensor of a motion detection system, the motion detection sensors may be arranged on a substrate (or a substrate having the motion detection sensors arranged thereon may be arranged) such that the sensing elements are orientated lengthwise vertical in order to detect horizontal motion in the ambient of the system.

In the alternative configuration shown in FIG. 4 in which a quad set of sensing elements is employed in each PIR sensor of PIR-based motion detection sensors 22 and 24, substrate 26 may be arranged (or PIR-based motion detection sensors 22 and 24 may be arranged on substrate 26) such that sensing elements 38 and 39 are oriented with their sides parallel with horizontal and vertical planes of the ambient of motion detection system 20. In other words, sensing elements 38 and 39 may be oriented as squares as shown in FIG. 4 as opposed to being oriented as diamonds (i.e., being tilted relative to the horizontal and vertical planes of the ambient of motion detection system 20). It is noted that a quad set of sensing elements may be configured to detect motion along orthogonal planes in the ambient of the motion detection system and, as such, in embodiments in which motion detection system 20 is configured to detect motion along a horizontal plane, the system can be configured to detect motion along a vertical plane as well.

In any case, in embodiments in which motion detection system 20 is configured to detect horizontal motion in an ambient of the system and PIR-based motion sensors 22 and 24 are vertically disposed, the optical systems of the motion sensors (i.e., Fresnel lens arrays 28 and 29) may be respectively configured such that a composite overlay of their horizontal fields of views has a beam pattern of one of the motion sensors overlapping a majority portion of dead zones of the other motion sensor or vice versa. In addition or alternatively, the different optical systems of PIR-based motion sensors 22 and 24 may be configured such that when PIR-based motion sensor 22 is arranged above PIR-based motion sensor 24 (or vice versa) detection zones associated with one of the PIR-based motion sensors have a higher density in a preset area view extending from its central axis than detection zones associated with the other PIR-based motion sensor in approximately the same preset area extending from its central axis and vice versa in different preset areas extending from the central axes. Example distributions of detection zones meeting such characteristics are described in detail below with respect to FIGS. 5-12.

Although horizontal movement is generally considered the most desirable direction for detecting motion and is specifically considered for the motion detection systems described herein when their pair of PIR-based motion detection sensors are vertically disposed, it is contemplated that the motion detection systems disclosed herein may be configured to detect motion along other directional planes, such as vertical planes or diagonal planes. In such cases, the PIR-based motion detection sensors 22 and 24 may be configured to increase the detection sensitivity of the motion detection system with respect to the plane along which movement is detected. For instance, instead of being vertically disposed, the pair of PIR-based motion detection sensors of the motion detection systems in such embodiments may be arranged laterally side-by-side in a horizontal direction (i.e., horizontally disposed for detecting motion along a vertical plane) or arranged catty-corner to each other (i.e., diagonally disposed for detecting motion along a diagonal plane). As an example, motion detection system 20 shown and described in reference to FIG. 1 may be turned 90° such that PIR-based motion detection sensors 22 and 24 are laterally arranged side-by-side (rather than vertically disposed). In yet other embodiments, motion detection system 20 may be turned less than 90° such that PIR-based motion detection sensors 22 and 24 are diagonally arranged.

In any of such cases, in order to increase the detection sensitivity of the motion detection system with respect to the plane along which movement is detected, the pair of PIR-based motion sensors may be arranged relative to each other such that central axes of their optical systems are substantially aligned with each other as taken from the viewpoint of the field of view in the plane along which motion is to be detected. In addition, the PIR-based motion detection sensors may, in some embodiments, be arranged in close proximity to each other, such as being spaced less than approximately 10 cm from each other. Furthermore, the optical systems of the PIR-based motion detection sensors may be configured to decrease the amount of dead zone area and/or increase the sensitivity in specific preset areas relative to their central axes in their vertical or diagonal field of views (as opposed to in their horizontal fields of view as referenced with respect to FIG. 1).

For example, the optical systems of the PIR-based motion detection sensors may be configured to affect a composite field of view having detection zones of one motion sensor overlapping dead zones of the other motion sensor or vice versa as taken along the vertical plane or diagonal plane which motion is to be detected. Alternatively stated, in relation to the plane along which motion is to be detected by the motion detection systems described herein, the pair of PIR-based motion sensors of the systems may be specifically configured such that a composite overlay of their fields of view in that plane has a beam pattern of one of the motion sensors overlapping a majority portion of dead zones of the other motion sensor or vice versa. In particular, the pair of PIR-based motion sensors include optical systems of respectively different configurations to yield the noted composite overlay of their detection zones and dead zones.

In addition or alternatively, the optical systems of horizontally disposed or diagonally disposed PIR-based motion detection sensors may be configured to affect density variation of detection zones in a field of view along the vertical plane or diagonal plane which motion is to be detected. More specifically, the pair of PIR-based motion sensors may be configured such that detection zones associated with one of the PIR-based motion sensors have a higher density in a preset area extending from its central axis than detection zones associated with the other PIR-based motion sensor in approximately the same preset area extending from its central axis and vice versa in different preset areas extending from the central axes. Layouts of detection zones and dead zones embodying such fields of view for horizontally disposed or diagonally disposed PIR-based motion detection sensors may be similar to what is described below in FIGS. 5-12 for systems having vertically disposed PIR-based motion detection sensors and which are configured to detect motion along a horizontal plane.

Turning to FIGS. 5-12, example distributions of detection zones of Fresnel lens arrays 28 and 29 are shown. In particular, FIGS. 5-7 respectively illustrate a vertical field of view, a horizontal field of view, and a cross-sectional field of view of an example distribution of detection zones of Fresnel lens array 28. In addition, FIGS. 8-10 respectively illustrate a vertical field of view, a horizontal field of view, and a cross-sectional field of view of an example distribution of detection zones of Fresnel lens array 29. Moreover, FIGS. 11 and 12 respectively illustrate a composite overlay of the detection zones of the horizontal fields of view of FIGS. 6 and 9 and a composite overlay of the detection zones of cross-sectional fields of view of FIGS. 7 and 10. It is noted that the vertical fields of view shown in FIGS. 5 and 8 are respectively taken from a side view of PIR-based motion detection sensors 22 and 24 when they are vertically disposed along substrate 26 as depicted in FIG. 1 and oriented on substrate 26 as shown in FIG. 3A. Furthermore, the horizontal fields of view shown in FIGS. 6 and 8 are respectively taken from a top view of PIR-based motion detection sensors 22 and 24 when they are vertically disposed along substrate 26 as depicted in FIG. 1 and oriented on substrate as shown in FIG. 2A. Moreover, as respectively shown by cross-hatched lines B and C in FIGS. 5, 6, 8, and 9, the cross-sectional fields of view shown in FIGS. 7 and 10 are respectively taken 5 and 7 meters from each of PIR-based motion detection sensors 22 and 24 along axes 30 and 32 when the motion detection sensors are vertically disposed along substrate 26 as depicted in FIG. 1 and oriented on substrate as shown in FIGS. 2A and 3A.

The fields of view of FIGS. 5, 6, 8, 9 and 11 show the detection range of PIR-based motion detection sensors 22 and 24 as 10 meters and 12 meters, respectively, but smaller or larger detection ranges may be considered. In other embodiments, the detection ranges of PIR-based motion detection sensors 22 and 24 may be the same. As noted above, the term “detection range” as used herein refers to the maximum distance which a PIR-based motion detection sensor may reliably detect motion. As further noted above, a variance of detection ranges among PIR-based motion detection sensors 22 and 24 may allow motion to be detected at a relatively far distance in a given area from motion detection system 20 while also having relatively high sensitivity of detecting motion in a closer region in the same given area. As noted above, the sensitivity of a PIR sensor decreases with distance from the sensor due to the inverse square law. As such, PIR-based motion detection sensor 24 having the longer detection range will be able to detect movement at a farther distance from motion detection system 20 but with less sensitivity than PIR-based motion detection sensor 22. However, the lower detection sensitivity of PIR-based motion detection sensor 24 may be compensated by the higher detection sensitivity of PIR-based motion detection sensor 22 making the field of view of motion detection 20 have a relatively high sensitivity to motion detection at least within the detection range of PIR-based motion detection sensor 22.

The field of views of FIGS. 5 and 8 show the vertical detection angles of PIR-based motion detection sensors 22 and 24 substantially similar and each approximately 120°, but smaller or larger as well as dissimilar detection angles may be considered. The field of views of FIGS. 6, 9 and 11 show the horizontal detection angles of PIR-based motion detection sensors 22 and 24 substantially similar and each approximately 110°, but smaller or larger as well as dissimilar detection angles may be considered. Since PIR-based motion detection sensors 22 and 24 are arranged such that their sensing elements 38 and 39 are configured to detect horizontal movement in ambient of motion detection system 20, the distribution of detection zones and dead zones of the horizontal fields of views in FIGS. 6, 9 and 11 are emphasized below, particularly with respect to increasing the sensitivity of detecting horizontal movement. The distribution of detection zones and dead zones of the vertical and cross-sectional fields of view of the optical systems of PIR-based motion detection sensors 22 and 24, however, are shown for reference in FIGS. 5, 7, 8, 10 and 12.

As described above, the vertical arrangement of PIR-based motion detection sensors 22 and 24 and an arrangement of sensing elements of their PIR sensors to detect motion along a horizontal plane enables a composite overlay of the horizontal field of views of the optical systems to represent a horizontal field of view of the motion detection system 20 as whole. As further described above, Fresnel lens arrays 28 and 29 are different and specifically configured, with respect to their horizontal field of view, to decrease the amount of dead zone area and/or increase the sensitivity in specific preset areas relative to their central axes to increase the detection sensitivity of motion detection system 20 as a whole as compared to using one or multiple PIR-based motion detection sensors with the same optical system configuration. In particular, as discussed in more detail below and shown in FIGS. 11 and 12 compared to FIGS. 6, 7, 9 and 10, fields of view of Fresnel lens arrays 28 and 29 facilitate composite fields of views having detection zones of PIR-based motion detection sensor 22 overlapping dead zones of PIR-based motion detection sensor 24 and vice versa. In addition, the example horizontal fields of view of respective Fresnel lens arrays 28 and 29 illustrated in FIGS. 6 and 9 have a variation in density of detection zones among PIR-based motion detection sensors 22 and 24 which increase detection sensitivity relative to what is offered by conventional systems.

Such characterizations of the detection zones of Fresnel lens arrays 28 and 29 are respectively described in reference to the right and left sides of their horizontal fields of view depicted in FIGS. 6 and 9, particularly relative to central axes 30 and 32. The characterizations, however, are not limited to half of the horizontal fields of view. Rather, the characterizations of the detection zones of Fresnel lens arrays 28 and 29 provided below with respect to detection zones 42 and 52, dead zones 44 and 54, as well as areas 46-48 and 56-58 may extend to the opposing side of central axes 30 and 32 as shown in FIGS. 6 and 9. Furthermore, it is noted that the distribution of detection zones and dead zones depicted in FIGS. 5-12 are examples. Other distributions of detection zones and dead zones may be considered which yield a composite overlay of fields of views having detection zones of one PIR-based motion detector sensor overlapping a majority of the dead zones of another PIR-based motion detector sensor and/or have a density variation of detection zones extending from their central axes.

As shown in FIG. 6, the horizontal field of view of the optical system of PIR-based motion detection sensor 22 (i.e., Fresnel lens array 28) has beam pattern 40 having a plurality of cross-hatched detection zones 42 extending to areas on either side of central axis 30. Conversely, the horizontal field of view of the optical system of PIR-based motion detection sensor 24 (i.e., Fresnel lens array 29) has beam pattern 50 having a plurality of cross-hatched detection zones 52 extending to areas on either side of central axis 32 as shown in FIG. 9. The term “detection zone” as used herein refers to portions of an ambient area of an optical system from which the optical system is configured to receive infrared radiation. When a person or object passes through two or more of plurality of detection zones 42 or 52, the associated PIR sensor will process the differential change in infrared radiation and generate an output signal corresponding to the level of change. Interspersed between plurality of detection zones 42 and 52 are “dead zones” 44 and 54 respectively for Fresnel lens arrays 28 and 29. The term “dead zone” as used herein refers to portions of an ambient area of an optical system within its field of view which the optical system does not receive infrared radiation (i.e., areas of the field of view in which detection is not possible).

As shown in FIG. 11, a composite overlay of the horizontal field of views of FIGS. 6 and 9 (i.e., as taken from a top view of motion detection system 20) has detection zones 42 of beam pattern 40 overlapping a majority portion of dead zones 54. In addition, FIG. 11 shows the composite overlay having detection zones 52 of beam pattern 50 underlying a majority portion of dead zones 44 as a whole. The term “majority”, as used herein, refers to greater than 50%. Further yet, the composite overly shown in FIG. 11 illustrates the dead zone area in the horizontal field of view of motion detection system 20 is much smaller than embodiments in which the motion detection system utilizes one of PIR-based motion detection sensors 22 and 24 or has PIR-based motion detection sensors 22 and 24 arranged in a different manner (i.e., not vertically disposed and/or not having the central axes of Fresnel lens arrays 28 and 29 substantially aligned). Alternatively stated, the relative arrangement of PIR-based motion detection sensors 22 and 24 and the differing configuration of their Fresnel lens arrays 28 and 29, particularly to induce an overlap of detection zones of one motion detection sensor over dead zones of another or vice versa, results in a higher concentration of detection zones in the field of view of motion detection system 20 as is shown in both FIGS. 11 and 12. As a result, the detection sensitivity of motion detection system 20 is increased relative to what is offered by conventional systems.

As shown in FIG. 6, the size and distribution of detection zones 42 and dead zones 44 of Fresnel lens array 28 are not uniform, particularly that detection zones 42 are arranged to have a dense area of side detection. Furthermore, as shown in FIG. 9, the size and distribution of detection zones 52 and dead zones 54 of Fresnel lens array 29 are not uniform, particularly that detection zones 52 are arranged to have a dense area of central detection (i.e., about axis 32) and some side detection as well. In addition, dead zones 54 includes a dead zone along and extending from central axis 32. The difference in the respective configurations of Fresnel lens arrays 28 and 29 to have such a variation in distributions of detection zones may be advantageous for ensuring the detection zones 42 of PIR-based motion detection sensor 22 overlap a majority portion of dead zones 54 of PIR-based motion detection sensor 24 or vice versa. In particular, it may be beneficial for one or both of beam patterns 40 and 50 to be non-uniform and have concentrated distributions of detection zones such that the overlap of detection zones and dead zones of PIR-based motion detection sensors 22 and 24 may be more easily attained. Conversely, in some embodiments, at least portions of beam patterns 40 and 50 may be uniform and arranged such that the composite overlay in the horizontal field of view has a sequence of beams which alternate between beams of beam pattern 40 and beams of beam pattern 50.

As noted above, in addition to producing the composite overlay of horizontal fields of view shown in FIG. 11, the example horizontal fields of views of respective Fresnel lens arrays 28 and 29 illustrated in FIGS. 6 and 9 have a variation in density of detection zones among PIR-based motion detection sensors 22 and 24 which increase detection sensitivity relative to what is offered by conventional systems. In particular, the horizontal field of view shown in FIG. 6 embodies area 46 extending from central axis 30 to angle Φ and area 47 extending from angle Φ to angle Ψ. In addition, the horizontal field of view shown in FIG. 9 embodies area 56 extending from central axis 32 to the same angle Φ and area 57 extending from angle Φ to angle Ψ. As shown in FIGS. 6 and 9, area 56 includes a higher density of detection zones than area 46 and area 47 includes a higher density of detection zones than area 57. In alternative embodiments, the relative variation of detection zone density among areas 46, 47, 56 and 57 may be reversed (i.e., area 46 may include a higher density of detection zones than area 56 and area 57 may include a higher density of detection zones than area 47). In any case, the countering detection zone density among areas 46, 47, 56 and 57 yields a higher concentration of detection zones in the horizontal field of view of motion detection system 20 as is shown in both FIGS. 11 and 12. As a result, the detection sensitivity of motion detection system 20 is increased relative to what is offered by conventional systems.

In some embodiments, areas 46 and 47 depicted in FIG. 6 may respectively extend to angles Φ and Ψ from the opposing side of central axis 30, making preset areas 46 and 47 centered along central axis 30. Similarly, areas 56 and 57 depicted in FIG. 9 may respectively extend to angles Φ and Ψ from the opposing side of central axis 32, making areas 56 and 57 centered along central axis 32. In other embodiments, one or more of areas 46, 47, 56 and 57 may not be centered along their central axes and, in some cases, may not even extend along the opposing sides of their central axes. In any case, the horizontal fields of view shown in FIGS. 6 and 9 respectively embody one or more additional areas 48 and 58 extending from angle Ψ to respective boundaries 45 and 55 of their maximum detection angles. In some configurations, one or more additional areas 48 and 58 may have a variation of detection zone density relative to each other as do areas 46 and 56 or as do areas 47 and 57 (i.e., one or more additional areas 48 may include a higher density of detection zones than one or more additional areas 58 or vice versa).

It is noted that although distributions of detection zones shown in the horizontal fields of views of FIGS. 6 and 9 have a density variation of detection zones extending from their central axes and also yield a composite overlay of horizontal fields of views having detection zones of one PIR-based motion detector sensor overlapping a majority of the dead zones of another PIR-based motion detector sensor, the motion detection systems disclosed herein are not necessarily restricted to having a pair of PIR-based motion detection sensors which yield both characterizations of detection zone distributions. In particular, the motion detection systems disclosed herein may include a pair of PIR-based motion detection sensors which yield one of the noted characterizations of detection zone distributions, but not the other. For instance, a motion detection system disclosed herein may include a pair of PIR-based motion detection sensors having a density variation of detection zones extending from their central axes, but not yield a composite overlay of horizontal fields of views having detection zones of one PIR-based motion detector sensor overlapping a majority of the dead zones of another PIR-based motion detector sensor. Conversely, a motion detection system disclosed herein may include at least two PIR-based motion detection sensors yielding a composite overlay of horizontal fields of views having detection zones of one PIR-based motion detector sensor overlapping a majority of the dead zones of another PIR-based motion detector sensor, but not have a density variation of detection zones extending from their central axes.

As noted above, the motion detection systems described herein include detection circuitry independently and operatively coupled to its PIR-based motion detection sensors. Detection circuitry 31 of motion detection system 20 is shown embedded within substrate 26 in FIGS. 2A-3C behind the PIR sensors of PIR-based motion detection sensors 22 and 24. In general, the detection circuitry of the motion detection systems described herein is configured to produce a motion detection signal upon receipt of a threshold signal from either PIR-based motion detection sensor that indicates a predetermined change of infrared radiation has been detected at the respective PIR-based motion detection sensor. Such a configuration of the detection circuitry makes the motion detection system highly sensitive to movement in the fields of view of the PIR-based motion detection sensors since motion detected only by one of the sensors (rather than both) is needed to affect operation of a device operationally coupled thereto. The phrase “predetermined change of infrared radiation” corresponds to a preset amount of differential change in infrared radiation and, thus, sets a threshold level of infrared radiation change by which to indicate that motion has been detected.

As discussed above with respect to motion detection system 20 of FIGS. 1-12, when a person or object passes into one or more of plurality of detection zones 42 or 52 (depending on the number of sensing elements of the PIR sensors of PIR-based motion detection sensors 22 and 24), the associated PIR sensor will process the differential change in infrared radiation and generate an output signal corresponding to the level of change. If that output signal corresponds to a level of infrared radiation which exceeds the predetermined change of infrared radiation, the associated PIR sensor will generate a threshold signal to send to the detection circuitry of the motion detection system or the detection circuitry itself will evaluate the output signal of the associated PIR sensor to determine whether it is a threshold signal (i.e., whether the output signal corresponds to a level of infrared radiation which exceeds the predetermined change of infrared radiation).

In either case, upon receipt of a threshold signal from either of the PIR-based motion detection sensors, the detection circuitry produces a motion detection signal and sends the motion detection signal to a device to affect its operation. Alternatively stated, the detection circuitry of the motion detection systems described herein is independently and operatively coupled to each of the PIR-based motion detection sensors such that upon receipt of a threshold signal from either of the motion detection sensors a motion signal may be generated and sent to affect operation of a device. In some embodiments, the device may be a visual or audible indicator which is configured to be activated in response to receiving of a motion detection signal from detection circuitry of a motion detection system, particularly for making notification that motion has been detected. In such cases, the device may be any changeable indicator known in the art, such as but not limited to a lighted bulb, an audible alarm and/or a visual display, the latter of which may include but is not limited to text on a graphical user interface. In other embodiments, the device may be a component of an apparatus or system which is configured to be activated or controlled in response to receiving of a motion detection signal from detection circuitry of a motion detection system. Examples of components may include but are not limited to electronic devices connected to a security system, a room occupancy system, and/or a home automation system (e.g., overhead lighting). Other examples of components may include but are not limited to a germicidal source of a room disinfection system (such as described in more detail below in reference to FIGS. 14-16) and an x-ray source of x-ray computed tomography equipment.

In some cases, the detection circuitry of the motion detection systems described herein may be configured for redundancy, particularly including multiple logic devices and timing and signal chains coupled to each of the PIR-based motion detection sensors. In general, detection circuitry configured for redundancy increases the reliability of a motion detection system to detect motion in an area. In particular, the redundancy permits the motion detection system to detect motion even if one of the logic devices, timing and signal chains, or PIR sensors is damaged or fails to operate. FIG. 13 illustrates a schematic layout of an example of redundant circuitry independently coupled between two PIR motion detection sensors and a system using motion detection signals generated by the circuitry. In particular, FIG. 13 depicts detection circuitry 31 of motion detection system 20 independently coupled between PIR-based motion detection sensors 22 and 24 and device 60. As set forth above, device 60 may be a visual or audible indicator which is configured to be activated in response to receiving of a motion detection signal from detection circuitry of a motion detection system or may be a component of an apparatus or system which is configured to be activated or controlled in response to receiving of a motion detection signal from detection circuitry of a motion detection system.

As shown, detection circuitry 31 includes logic devices 62 and 64 individually coupled to each of PIR-based motion detection sensors 22 and 24 to receive output signals therefrom. In addition, detection circuitry 31 includes timing and signal chains 66 and 68 respectively coupled to logic devices 62 and 64. In general, logic devices 62 and 64 evaluate output signals from PIR-based motion detection sensors 22 and 24 to determine whether a received output signal is a threshold signal indicating motion has been detected by the respective PIR-based motion detection sensor. Upon determining a received output signal is a threshold signal, logic devices 62 and 64 respectively transmit a signal to timing and signal chains 66 and 68 to generate a motion detection signal and affect the timing of its transmittal to device 60.

As set forth above, motion detection system 20 and all of its variants may, in some embodiments, be used in conjunction with an apparatus or system. In some of such cases, substrate 26 may be a surface of the apparatus or system (i.e., substrate 26 may be part of the structure of the apparatus or system). In other embodiments, substrate 26 may be affixed to a surface of the apparatus or system (i.e., substrate 26 may be a distinct component from the apparatus or system but attached thereto). In yet other cases, motion detection system 20 may be a remote device but operationally coupled to the apparatus or system such that the motion detection system may be used to affect operation of the apparatus or system. In any case, systems and apparatuses having motion detection system 20 (or any variation thereof described above) may be portable or may be fixedly arranged in a room. In some cases, a system or apparatus may have multiple PIR-based motion detection systems disposed along different sides of the apparatus for detecting motion in distinct areas extending different directions from the apparatus. In some of such cases, the multiple motion detection systems may each be a PIR-based motion detection multiple sensor coalesced beam pattern system as described for motion detection system 20 (or any variants thereof) in reference to FIG. 1. As set forth above, examples of systems and apparatuses having motion detection system 20 (or any variation thereof described above) include but are not limited to area/room disinfection apparatuses, security systems, home automation systems, and room occupancy monitoring systems, particularly ones that are used to prevent exposure to hazardous conditions, such as but not limited to systems for rooms containing X-ray computed tomography equipment.

Further to the disclosure of apparatuses and systems having a motion detection system with at least two PIR-based motion detection sensors which are vertically disposed, arranged such that the central axes of their optical systems are substantially aligned, and/or in relatively close proximity to each other (as described in reference to motion detection system 20), portable apparatuses having at least two PIR-based motion detection sensors with different detection ranges are also disclosed herein. The different characterizations of apparatuses may be inclusive to a single apparatus or may be directed at different apparatuses. In particular, as noted above, motion detection system 20 may include a pair of PIR-based motion detection systems having different detection ranges, but in other cases their detection ranges may be the same. In addition, systems and apparatuses having motion detection system 20 (or any variation thereof described above) may be portable or may be fixedly arranged in a room.

Moreover, portable apparatuses are disclosed herein that include at least two PIR-based motion detection sensors having different detection ranges, but are not vertically disposed, are not arranged such that the central axes of their optical systems are substantially aligned, and/or are not in relatively close proximity to each other. For instance, the motion detection systems of some of the portable apparatuses considered herein may include PIR-based motion detection sensors disposed laterally side-by-side. In addition or alternatively, the motion detection systems of some of the portable apparatuses considered herein may have PIR-based motion detection sensors individually arranged at respectively different locations of the apparatuses, particularly spaced apart such that each sensor is disposed on its own printed circuit board. For instance, the motion detection systems of some of the portable apparatuses considered herein may have PIR-based motion detection sensors having different detection ranges that are disposed along different sides of the apparatus for detecting motion in distinct areas extending different directions from the apparatus. A variance of detection ranges among motion detection sensors may be advantageous when a portable apparatus is to be utilized in relatively large rooms having non-uniform dimensions. In particular, having at least two PIR-based motion detection sensors arranged along different sides of a portable apparatus enables the portable apparatus to be positioned in a room such that the motion detection sensor having the larger detection range is oriented to monitor an area in a larger dimension of the room and the motion detection sensor having the smaller detection range is oriented to monitor an area in a smaller dimension of the room.

As used herein, the terms “mobile” and “portable” refer to the capability of moving or being moved and may be used interchangeably herein. Configurations to affect mobility or portability of some of the apparatuses considered herein may include but are not limited to wheels (motorized or non-motorized), one or more handles, navigational program instructions (including preprogrammed paths, navigation via remote control and/or autonomous capability), a weight and design which affords the apparatus to be efficiently and safely transported at least 1 meter, or any combination thereof. It is emphasized that the apparatuses considered herein may include any one or more of such configurations in any combination to affect their mobility and, thus, the apparatuses are not limited to including all of the noted configurations. For instance, an apparatus which is motorized may or may not include navigational program instructions. Furthermore, an apparatus configured for autonomous movement may or may not include a handle. Moreover, an apparatus having one or more handles may or may not have wheels and vice versa. In addition, an apparatus with wheels is generally easier to push or pull than an apparatus without wheels and, thus, apparatuses considered herein having wheels may be but are not necessarily heavier than those without wheels.

In general, the parameters constituting “a weight and design which affords the apparatus to be efficiently and safely transported at least 1 meter” may vary among apparatuses. For instance, in cases in which an apparatus is not motorized, the phrase may pertain to an ergonomic weight and design which affords the apparatus to be efficiently and safely carried, pushed and/or pulled at least 1 meter by one or more adults of average height and weight. For example, the weight of an apparatus considered herein that is not motorized may be less than approximately 25 pounds, particularly but not limited to if the apparatus does not include wheels and is of a design (i.e., size, shape, etc.) that one individual may manipulate the relocation of the apparatus. Conversely, the weight of an apparatus considered herein that is not motorized may, in some cases, be more than approximately 25 pounds but less than approximately 200 pounds, particularly if the apparatus includes wheels and/or is of a design that facilitates for multiple individuals to manipulate the relocation of the apparatus (e.g., includes multiple handles). In yet other embodiments, the weight of an apparatus considered herein that is not motorized and has wheels and/or has a design that multiple individuals may manipulate the relocation of may be less than approximately 25 pounds.

In alternative embodiments, some of the portable apparatuses considered herein may be motorized and, in such cases, the weight of the portable apparatus may not be as restricted as those which are not motorized, particularly an apparatus which is motorized may be any weight and design which affords the apparatus to efficiently and safely travel at least 1 meter. For example, an apparatus considered herein that is motorized may be of a weight and have motor controls which allow the movement of the apparatus to be started and stopped without undue time delay (e.g., less than 5 seconds). In addition, an apparatus considered herein that is motorized may be of a weight and include speed controls, navigational controls and/or guarding which allows the apparatus to be moved without causing substantial damage to the apparatus or infrastructure along the path of the apparatus. For instance, an apparatus considered herein that is motorized may include controls which prevent the apparatus from bombarding with obstacles and/or walls. In addition or alternatively, an apparatus considered herein that is motorized may be configured to limit the speed of the apparatus, have bumpers along an outermost periphery of the apparatus and/or have fragile components arranged in protective housings and/or arranged inward from an outermost periphery of the apparatus. Further to the idea of the apparatuses considered herein being configured to efficiently travel at least 1 meter, the speed controls of a motorized apparatus may be sufficient such that an apparatus may travel to a position in a timely manner. An example range of speed for the apparatuses considered herein may be up to approximately 200 meter/minute, but faster speeds may be considered.

In any case, an apparatus that is motorized may include configurations for a user to guide the apparatus and/or configurations for the apparatus to autonomously guide itself. Example configurations which may enable a user to guide a mobile apparatus include one or handles and/or one or more user input controls, such as but not limited to a steering wheel, a joystick, a means for enabling audible input for directional movement and/or one or more tactile input controls denoting particular directional movements (such as but not limited to buttons, switches, graphical user interfaces and/or touch sensor means). In some cases, configurations for enabling a user to guide an apparatus may be integrated into the apparatus such that movement of the apparatus may be controlled at the apparatus. In yet other cases, user input controls for enabling a user to guide an apparatus may be integrated into a detached user interface used in conjunction with the apparatus such that movement of the apparatus may be controlled via remote control. In some cases, a mobile apparatus may include configurations integrated into the apparatus and configurations integrated into a detached user interface for enabling a user to guide its movement. In yet other cases, a mobile apparatus may be additionally or alternatively configured to guide itself, i.e., via program instructions to follow a predetermined path and/or via navigational controls for autonomous movement.

In some embodiments, a mobile apparatus considered herein may have a maximum height of approximately 75 inches and/or a maximum width of approximately 70 inches, and in particular cases, a maximum width of approximately 32 inches. In particular, such size restrictions may be advantageous in embodiments in which a mobile apparatus is to be used in different spaces in a building some of which have limited access for entry, such as through a door or a passageway having the width of a door. More specifically, in order to accommodate the use of a mobile apparatus in such spaces, the mobile apparatuses disclosed herein may have size limitations such that they may be moved into the spaces through their entryways. In other cases, a mobile apparatus considered herein may be used in spaces of a building which are not limited by an entry having a door frame or a passageway of similar dimensions and, thus, the mobile apparatuses used in such spaces may not have the aforementioned width and/or height limitations.

Turning to FIGS. 14 and 15, example configurations of disinfection apparatuses are shown, each having a motion detection system with at least two PIR-based motion detection sensors. In particular, FIG. 14 illustrates disinfection apparatus 70 having PIR-based motion detection multiple sensor coalesced beam pattern systems 72 and 74 and FIG. 15 illustrates disinfection apparatus having PIR-based motion detection sensors 82, 83 and 84. As shown, disinfection apparatus 70 includes a single germicidal light source 76 and, in contrast, disinfection apparatus 80 includes plurality of germicidal light sources 86. The number and dimensions of germicidal light sources making up each disinfection apparatus is not mutually exclusive to the configuration of each apparatus, particularly to the other notable differences between the disinfection apparatuses (such as but not limited to the number and configuration of their motion detection systems and the shape and size mobile carriages 78 and 88 supporting the germicidal sources of each apparatus). As such, disinfection apparatus 70 may alternatively have multiple germicidal light sources and/or may have a germicidal light source with a different length to width ratio than shown in FIG. 14. Furthermore, disinfection apparatus 80 may alternatively have a single germicidal light source and/or may have one or more germicidal light sources with a different length to width ratio than shown in FIG. 15.

The term “germicidal light”, as used herein, refers to light which is capable of deactivating or killing microorganisms, particularly disease carrying and/or disease producing microorganisms (a.k.a., germs). Ranges of light which are known to be germicidal include ultraviolet B (UVB) and ultraviolet C (UV-C) light, particularly ultraviolet light between approximately 200 nm and approximately 320 nm, and more particularly ultraviolet light at 220 nm and ultraviolet light between 260 nm and 280 nm. Another range of light which is known to be germicidal includes visible violet-blue light (also known as high-intensity narrow-spectrum (HINS) light) between approximately 400 nm and approximately 470 nm, and particularly at 405 nm. The germicidal sources considered for the disinfection apparatuses disclosed herein may be configured to generate any one or more of such ranges or wavelengths of germicidal light.

In some embodiments, a germicidal light source of the disinfection apparatuses considered herein may generate ranges of light which are not germicidal such as but not limited to visible light greater than approximately 500 nm, but such capability will not deter from the reference of the light source being germicidal. To that regard, a light source or lamp of the disinfection apparatuses disclosed herein may, in some cases, be characterized in the type of light it generates, but such characterization need not limit the light source or lamp to generating only that type of light. For example, an ultraviolet lamp is one which generates ultraviolet light, but it may produce light of other wavelengths. In any case, the germicidal light sources considered for the disinfection apparatuses described herein may be of any size and shape, depending on the design specifications of the disinfection apparatuses. The terms “germicidal light source” and “germicidal lamp” are used interchangeably herein and refer to a collection of one or more components used to generate and disperse germicidal light.

Examples of germicidal light sources which may be configured to generate ultraviolet light and/or HINS light include discharge lamps, light emitting diode (LED) solid state devices, and excimer lasers. HINS lamps are generally constructed of LEDs. A discharge lamp as used herein refers to a lamp that generates light by means of an internal electrical discharge between electrodes in a gas. The term encompasses gas-discharge lamps, which generate light by sending an electrical discharge through an ionized gas (i.e., a plasma). The term also encompasses surface-discharge lamps, which generate light by sending an electrical discharge along the surface of a dielectric substrate in the presence of a gas, producing a plasma along the substrate's surface. As such, the discharge lamps which may be considered for the germicidal sources described herein include gas-discharge lamps as well as surface-discharge lamps.

Discharge lamps may be further characterized by the type of gas/es employed and the pressure at which they are operated. The discharge lamps which may be considered for the germicidal sources described herein may include those of low pressure, medium pressure, and high intensity. In addition, the gas/es employed may include helium, neon, argon, krypton, xenon, nitrogen, oxygen, hydrogen, water vapor, carbon dioxide, mercury vapor, sodium vapor and any combination thereof. In some embodiments, various additives and/or other substances may be included in the gas/es. In any case, the discharge lamps considered for the germicidal sources described herein may include those which generate continuous light and those which generate light in short durations, the latter of which are often referred to as flashtubes or flashlamps. Flashtubes or flashlamps that are used to supply recurrent pulses of light are often referred to as pulsed light sources.

A commonly used gas-discharge lamp used to produce continuous light is a mercury-vapor lamp (which is inclusive of mercury only lamps as well as mercury amalgam lamps), which may be considered for the disinfection apparatuses described herein. It emits a strong peak of light at 253.7 nm, which is considered particularly applicable for germicidal disinfection and, thus, is commonly referenced for ultraviolet germicidal irradiation (UVGI). A commonly used flashlamp which may be considered for the mobile disinfection apparatuses described herein is a xenon flashtube. In contrast to a mercury-vapor lamp, a xenon flashtube generates a broad spectrum of light from ultraviolet to infrared and, thus, provides ultraviolet light in the entire spectrum known as germicidal (i.e., between approximately 200 nm and approximately 320 nm). In addition, a xenon flashtube can provide relatively sufficient intensity in the spectrum which is known to be optimally germicidal (i.e., between approximately 260 nm and approximately 265 nm). Moreover, a xenon flashtube generates an extreme amount of heat, which can further contribute to the deactivation and killing of microorganisms. It is noted that the aforementioned descriptions and comparisons of a mercury-vapor lamp and a xenon flashlamp in no way restrict the disinfection devices described herein to include such lamps. Rather, the aforementioned descriptions and comparisons are merely provided to offer factors which one skilled in the art may contemplate when selecting a germicidal light source for the disinfection apparatuses described herein.

As noted above, in some cases, the germicidal light source may be an excimer laser and, thus, the germicidal light projected into an ambient of the disinfection apparatuses disclosed herein may be a narrow beam of light. In some cases, a disinfection apparatus having a laser may be configured to move the laser and/or the apparatus may be configured to move itself during a disinfection cycle such that multiple surfaces may be disinfected by the laser. The disinfection apparatus may be of any shape, size, or configuration in which to achieve such an objective.

In some embodiments, disinfection apparatus 70 and/or disinfection apparatus 80 may include a germicidal source other than a germicidal light source (i.e., in addition or alternative to germicidal light sources 76 and 86). In particular, the germicidal sources considered herein for the disinfection apparatuses disclosed therein may be any device configured to generate a dispersible germicide. More specifically, the germicidal sources considered herein may be any device or apparatus configured to generate a germicide in form of a liquid, a vapor, a gas, a plasma, or germicidal light. Examples of germicidal sources which may be configured to disperse liquid, vapor, gaseous, or plasma germicides include but are not necessarily limited to liquid sprayers, foggers, plasmas torchers and misting systems including wet and dry mist systems. An example of a gaseous germicide is ozone. Examples of plasmas germicides include reactive oxygen species. Examples of liquid and vapor germicides include liquid and vapor disinfection solutions having a principal disinfection agent such as but not limited to bleach, hydrogen peroxide, chlorine, alcohol, or quaternary ammonium compounds.

As used herein, the term “germicide” refers to an agent for deactivating or killing microorganisms, particularly disease carrying and/or disease producing microorganisms (a.k.a., germs). The term “kill,” as used herein, means to cause the death of an organism. In contrast, the term “deactivate,” as used herein, means to render an organism unable to reproduce without killing. As such, a germicide which is configured to deactivate a microorganism, as used herein, refers to an agent which renders a microorganism unable to reproduce but leaves the organism alive. Furthermore, the term “germicidal source” as used herein refers to a collection of one or more components used to generate and disperse a germicide. In some embodiments, a germicidal source may include components in addition to the component/s used to generate the germicide to affect the dispersal of the germicide from the generation component/s.

It is noted that the disinfection apparatuses considered herein are not limited to apparatuses having a germicidal source extending beyond a support structure of the application and/or having a germicidal source arranged along the upper surface of a support structure as is shown for apparatuses 70 and 80 in FIGS. 14 and 15. In particular, a disinfection apparatus considered herein may, in some embodiments, have a germicidal source arranged partially or wholly inset to a structure of the apparatus with an opening or transparent window to allow the transmission of a germicide generated therefrom to be projected into an ambient of the apparatus. In addition, a disinfection apparatus considered herein may have a germicidal source arranged along any portion of the apparatus, including the top portion, sidewall portions or the bottom of the apparatus. Furthermore, the disinfection apparatuses described herein may include any number of germicidal sources. In general, the quantity, size, type and placement of germicidal sources on the disinfection apparatuses considered herein, including disinfection apparatuses 70 and 80 shown in FIGS. 14 and 15, may depend on the design specifications of the apparatus.

In any case, the disinfection apparatuses considered herein, including disinfection apparatuses 70 and 80, are emphasized in reference to area/room disinfection apparatuses and, as such, have their germicidal source/s arranged such that germicide is projected into an ambient of the apparatus (i.e., exterior to the apparatus). As used herein, the term “area/room disinfection apparatus” refers to an apparatus configured to disinfect a space which is suitable for human occupancy so as to deactivate, destroy or prevent the growth of disease-carrying microorganisms in the area. Furthermore, as used herein, the terms “area/room disinfection apparatus”, “area disinfection apparatus” and “room disinfection apparatus” may be used interchangeably herein. In addition to being configured to project germicide into an ambient of the apparatus, a disinfection apparatus may, in some embodiments, include one or more of the features described below that are generally associated with area/room disinfection apparatuses.

In general, an area/room disinfection apparatus includes configurations to distribute an effective amount of germicide in a spacious manner to an ambient of an area/room in which the apparatus is arranged to maximize the number of surfaces and objects disinfected in the area/room. The apparatus and/or any of its components may be of any shape, size, or configuration to achieve such an objective. For instance, vertically arranged germicidal source/s (e.g., having germicidal source/s arranged lengthwise substantially perpendicular to a horizontal plane of the apparatus) as shown for disinfection apparatuses 70 and 80 in FIGS. 14 and 15 may aid in distributing a germicide within an area/room. In particular, vertically arranged germicidal source/s may be advantageous for optimizing the vertical distribution of a germicide into an ambient of the apparatus and also distributing a germicide 360° around the apparatus. Despite such advantages, horizontally or diagonally arranged germicidal sources may be used in an area/room disinfection apparatus to distribute an effective amount of germicide in a spacious manner to an ambient of an area/room in which the apparatus is arranged.

Another configuration of an area/room disinfection apparatus which may be particularly considered for the disinfection apparatuses discussed herein and which is shown for disinfection apparatuses 70 and 80 in FIGS. 14 and 15 is to be configured to direct a germicide to a region approximately 2 feet and approximately 4 feet from a floor of an area/room in which the apparatus is arranged. In particular, the region between approximately 2 feet and approximately 4 feet from a floor of a room is considered a “high touch” region of an area/room since objects of frequent use are generally placed in such a region. Examples of configurations which offer such germicide direction are disclosed in U.S. patent application Ser. No. 13/706,926 filed Dec. 6, 2012 and Ser. No. 13/708,208 filed Dec. 7, 2012 and International Patent Application No. PCT/US 2014/059698 filed Oct. 8, 2014, all of which are incorporated herein by reference as if set forth fully herein. Other features specific to area/room disinfection apparatuses are disclosed in such documents as well. For example, other features of area/room disinfection apparatuses include wheels and/or a handle to affect portability for the apparatuses as is shown for disinfection apparatuses 70 and 80 in FIGS. 14 and 15. In alternative embodiments, disinfection apparatuses 70 and 80 may not include wheels and/or a handle. In any case, many area/room disinfection apparatuses include configurations for remotely starting the apparatuses such that individuals need not be present in the area/room when operation of the apparatus commences.

Another feature of an area/room disinfection apparatus which may be included in the disinfection apparatuses considered herein is to include configurations to distribute an effective amount of germicide to achieve at least a 2-log reduction in bacterial contamination on surfaces that are greater than 1 meter or even 2 or 3 meters from its germicidal source/s. In some cases, an area/room disinfection apparatus having such a configuration (i.e., configured is to distribute an effective amount of germicidal light to achieve at least a 2-log reduction in bacterial contamination on surfaces within a room or area that are greater than 1 meter or even 2 or 3 meters from the disinfection apparatus) may be referred to herein as a “whole room microbial reduction device.” Configurations used to generate such an effect generally depend on the configuration of the germicidal source/s, particularly the size of the germicidal source/s, the intensity and/or frequency at which the germicide is dispersed and/or the orientation of the germicidal source/s in the apparatus. For example, in cases in which an area/room disinfection apparatus includes one or more germicidal light sources, power fluxes of at least 1.0 W/m² may be generally used to achieve at least a 2-log reduction in bacterial contamination on surfaces within an area/room that are greater than 1 meter from the germicidal light source/s.

Furthermore, another feature common to area/room disinfection apparatuses which the disinfection apparatuses described herein may include is one or more actuators for moving its germicidal source/s with respect to other components of the apparatus (such as a base of the apparatus or a support structure supporting the germicidal source/s) to aid in the distribution of a germicide in an area/room. In such cases, a germicidal source may be moved in vertical, horizontal, and/or diagonal directions via the one or more actuators. For instance, disinfection apparatus 70 shown in FIG. 14 may, in some embodiments, include an actuator for moving germicidal light source 76 up and down while it is emitting light. It is noted that the configuration of an area/room disinfection apparatus to move one or more of its germicidal sources may include configurations to induce 360° distribution of a germicide around the apparatus as described below, but the apparatuses disclosed herein are not necessarily so limited. In particular, the area/room disinfection apparatuses disclosed herein may include configurations to induce any distribution of a germicide, including those which induce a distribution less than 360° around the apparatus. In some cases, the area/room disinfection apparatuses disclosed herein may include processor-executable program instructions for activating one or more of the actuator/s to move the germicidal source relative to other components of the apparatus while the germicidal source is emitting a germicide and also in between projections of the germicide in cases in which a pulsed germicidal source is used.

Another feature of an area/room disinfection apparatus which may be included in the disinfection apparatuses considered herein is a configuration to distribute a germicide 360° around the apparatus. For example, in some cases, the arrangement of one or more germicidal sources in a disinfection apparatus may be such that a germicide emitted from the germicidal source/s is projected approximately 360° around the apparatus. For instance and as is shown by germicidal light source 76 in FIG. 14, a disinfection apparatus may, in some cases, include a single germicidal source that is arranged to distribute a germicide approximately 360° around the apparatus. In other cases, a disinfection apparatus may include a plurality of germicidal sources that are arranged to collectively distribute a germicide approximately 360° around the apparatus, such as shown by germicidal light sources 86 in FIG. 15. In either of such cases, the disinfection apparatuses may be void of germicide-blocking components approximately 360° around their germicidal source/s such that a continuous ring of germicide is formed around the apparatus. In alternative cases, particularly in scenarios in which a disinfection apparatus includes a germicidal light source, the disinfection apparatus may include a reflector to form a continuous ring of germicidal light 360° around the apparatus.

In additional or alternative cases, a disinfection apparatus considered herein may be configured to move one or more of its germicidal source/s and/or one or more of its other components to distribute a germicide approximately 360° around the apparatus during a disinfection cycle of the apparatus. In such cases, the distribution of the germicide may not be a continuous ring of germicide around the apparatus but may be such that the germicide emitted from the germicidal source/s throughout a duration of a disinfection cycle collectively occupies the entirety of an encircling region around the apparatus. For instance, in some cases, a disinfection apparatus may include one or more moveable germicidal sources (such as but not limited to a sprayer or laser) that are configured to move to distribute a germicide 360° around the apparatus. In other cases, a disinfection apparatus may include a moveable housing around one or more of its germicidal sources, wherein the housing that has one or more openings or one or more transparent windows to transmit a germicide from the germicidal source/s to an exterior of the apparatus. In such cases, the housing and, in some cases, the germicidal source/s may be moved to achieve a 360° distribution of germicide around the apparatus through the openings and/or holes of the housing. In yet other cases, particularly in scenarios in which a disinfection apparatus includes a germicidal light source, the disinfection apparatus may include a movable reflector to help distribute germicidal light 360° around the apparatus.

Yet another feature which may be included in the apparatuses described herein to specifically affect room/area disinfection is processor executable program instructions for receiving data regarding characteristics of an area/room in which the mobile disinfection apparatus is to be operated. In general, the phrase “characteristics of an area/room” as used herein refers to physical attributes as well as non-physical attributes of an area/room. Non-physical attributes of an area/room include but are not necessarily limited to identifiers used to reference an area/room (e.g., room number and/or room name) and occupancy information regarding an area/room (e.g., infection information of a patient previously occupying the space or a patient scheduled to occupy the space). Physical attributes of an area/room include but are not necessarily limited to size and/or dimensions of the area/room and/or the number, size, distances, locations, reflectivity and/or identification of surfaces, objects and/or items within the area/room. In some cases, a physical attribute of an area/room may be the identification of one or more pathological organisms and, sometimes further the number or concentration of such organism/s in the area/room, in a particular region of the area/room, or on a particular surface in the area/room.

In any case, the data received regarding the characteristics of the area/room in which the disinfection apparatus is to be operated may be utilized in a number of manners, including but not limited to setting one or more operational parameters of the apparatus or for recordation or reporting purposes. In some cases, the apparatuses described herein may include one or more sensors for detecting and/or mapping physical characteristics of an area/room and such information may be used for recordation and/or to affect operation of the apparatus. In additional or alternative cases, the apparatuses described herein may be programmed to access physical characteristics of an area/room from a database communicably coupled to the apparatus. For example, a preassigned room identifier (such as “103” or “Operating Room”) may be entered into an apparatus (such as by key entry at a user interface, scanning a barcode or receiving a wireless signal upon entry into the area/room) and one or more physical characteristics and, in some cases, a map or model of the area/room may be accessed from a database outlining such correlative information. As noted above, the apparatuses described herein may, in some cases, include a means for automatically moving the apparatus. In some such cases, the apparatus may include program instructions to move the apparatus in accordance with characteristics of an area/room. Examples of area/room disinfection apparatuses with some of the aforementioned program instructions are disclosed in U.S. application Ser. No. 13/706,926 filed Dec. 6, 2012, which is incorporated by reference as if set forth fully herein.

As set forth in more detail below, the motion detection systems of the disinfection apparatuses depicted in FIGS. 14 and 15 may, in some cases, include at least two PIR-based motion detection sensors having different detection ranges. As noted above, the term “detection range” as used herein refers to the maximum distance which a PIR-based motion detection sensor may reliably detect motion. As such, an alternative manner to describe the motion detection systems of the disinfection apparatuses depicted in FIGS. 14 and 15 is to say the motion detection systems of the apparatuses include at least two PIR-based motion detection sensors having different maximum detection distances. In yet other cases and as set forth below, the motion detection systems of the disinfection apparatuses depicted in FIGS. 14 and 15 need not include PIR-based motion detection sensors having different detection ranges. Rather, the motion detection systems of the disinfection apparatuses depicted in FIGS. 14 and 15 may alternatively include PIR-based motion detection sensors having substantially similar detection ranges.

As described in the example shown in FIG. 14, two PIR-based motion detection sensors with different maximum detection distances in a disinfection apparatus may, in some embodiments, be those of a PIR-based motion detection multiple sensor coalesced beam pattern system. In particular and as described above, a variance of detection ranges among two PIR-based motion detection sensors of a PIR-based motion detection multiple sensor coalesced beam pattern system may allow motion to be detected at a greater distance in a given area from the motion detection system while also having sensitivity of detecting motion in a closer region in the same given area. Despite such an advantage, a disinfection apparatus disclosed herein may alternatively have one or more PIR-based motion detection multiple sensor coalesced beam pattern systems with two PIR-based motion detection sensors having the same maximum detection distance. As such, the description of a disinfection apparatus having one or more PIR-based motion detection multiple sensor coalesced beam pattern systems is not limited to the description set forth below for disinfection apparatus 70 in reference to FIG. 14.

In other embodiments as described in reference to FIG. 15, PIR-based motion detection sensors having different maximum detection distances in a disinfection apparatus may not be vertically disposed relative to each other and/or may not be arranged such that their central axes are substantially aligned. For instance, a disinfection apparatus may include two or more PIR-based motion detection sensors having different maximum detection distances disposed along different sides of the apparatus for detecting motion in distinct areas extending different directions from the apparatus. Although an apparatus having two PIR-based motion detection sensors arranged in such a manner may not realize the benefit of detecting motion at a far distance but with relatively high sensitivity in a close region of the same given area as noted above for a PIR-based motion detection multiple sensor coalesced beam pattern system, the variance of detection ranges among the motion detection sensors may still be advantageous when the apparatus is portable and is to be utilized in relatively large rooms having non-uniform dimensions. In particular, such an arrangement of at least two PIR-based motion detection sensors having different maximum detection distances enables a portable apparatus to be positioned in a room such that the motion detection sensor having the larger detection range is oriented to monitor an area in a larger dimension of the room and the motion detection sensor having the smaller detection range is oriented to monitor an area in a smaller dimension of the room. Such a benefit may also be realized for a disinfection apparatus having multiple PIR-based motion detection multiple sensor coalesced beam pattern systems arranged on different sides of the disinfection apparatus.

In many uses of an area/room disinfection device, it is generally advantageous to be able to detect movement within the entirety of a room. For instance, portable area/room disinfection devices are often used in multiple rooms in a facility and the rooms are often of different sizes and dimensions. As an example, apparatuses configured for area/room disinfection are commonly used in a variety of rooms and areas of a hospital, including but not limited to single patient rooms, multiple occupancy patient rooms, bathrooms, walk-in closets, hallways, offices, operating rooms, patient examination rooms, waiting and/or lounging areas and nursing stations. Operating rooms, nursing stations and multiple occupancy patient rooms tend to be quite large, generally averaging over 600 square feet on average. In order to ensure movement can be detected throughout such rooms, it may be advantageous for the maximum detection distances of at least some motion detection systems on an area/room disinfection device to be greater than approximately 6 meters and, in some embodiments, greater than approximately 9 meters. Smaller or larger detection ranges, however, may be considered.

As described in more detail below, FIG. 14 illustrates disinfection apparatus 70 having PIR-based motion detection multiple sensor coalesced beam pattern systems 72 and 74 and FIG. 15 illustrates disinfection apparatus having PIR-based motion detection sensors 82, 83 and 84. In general, the quantity, size, type, and placement of the PIR-based motion detection sensors/systems on the disinfection apparatuses described herein may vary and may depend on the design specifications of the apparatus. As such, the quantity, size, type and placement of the PIR-based motion detection sensors/systems on disinfection apparatuses 70 and 80 are not restricted to what is depicted in FIGS. 14 and 15 and described in more detail below. For instance, disinfection apparatuses 70 and/or 80 may include more or less than the PIR-based motion detection sensors/systems shown.

Furthermore, although PIR-based motion detection multiple sensor coalesced beam pattern systems 72 and 74 may, in some embodiments, be arranged in proximity to an upper edge of mobile carriage 78 as shown in FIG. 14, one or both of them may be arranged along other portions of mobile carriage 78 or one or both of them may be arranged on a different component of disinfection apparatus 70 (such as but not limited to the handle of the apparatus or a reflector assembly disposed above germicidal light source 76 when such a reflector assembly is included in the apparatus). Similarly, although PIR-based motion detection sensors 82-84 may, in some embodiments, be arranged along cap assembly 89 as shown in FIG. 15, one or more of them may be arranged on a different component of disinfection apparatus 80 (such as but not limited to mobile carriage 88). In yet other embodiments, one or more of PIR-based motion detection multiple sensor coalesced beam pattern systems 72 and 74 and PIR-based motion detection sensors 82-84 may be remote from their respective disinfection apparatus, but operationally coupled thereto.

In some embodiments, the PIR-based motion detection sensors/systems of disinfection apparatus 70 and/or 80 may be configured to collectively monitor an area 360° around the apparatus for motion. More specifically, the number and relative arrangement of the PIR-based motion detection sensors of disinfection apparatus 70 and/or 80 may be such that motion can be detected approximately 360° around the apparatus. An example arrangement of PIR-based motion detection sensors configured to collectively monitor an area approximately 360° of motion detection around disinfection apparatus 70 would be to have one or more of PIR-based motion detection multiple sensor coalesced beam pattern systems 72 and 74 along each sidewall of rectangular mobile carriage 78, although other configurations of PIR-based motion detection sensors may be considered. An example arrangement of PIR-based motion detection sensors configured to collectively monitor an area approximately 360° of motion detection around disinfection apparatus 80 would be to have one or more of PIR-based motion detection sensors 82-84 circumferentially arranged along cap assembly 89 and/or along mobile carriage 88, although other configurations of PIR-based motion detection sensors may be considered. An example arrangement of PIR-based motion detection sensors along sidewalls of an oblong apparatus as taken from a top view of the apparatus is shown in FIG. 16 and described in more detail below.

As noted above, FIG. 14 illustrates disinfection apparatus 70 having PIR-based motion detection multiple sensor coalesced beam pattern systems 72 and 74, which together may completely or partially make up a motion detection system for the disinfection apparatus. In general, PIR-based motion detection multiple sensor coalesced beam pattern systems 72 and 74 may each include two PIR-based motion detection sensors which are vertically disposed such that the central axes of their optical systems are substantially aligned as is described for PIR motion detection sensors 22 and 24 of motion detection system 20 discussed above in reference to FIGS. 1-13. In some of such cases, one or both of PIR-based motion detection multiple sensor coalesced beam pattern systems 72 and 74 may include additional configurations described above for motion detection system 20, including any mentioned variations which are not depicted in the drawings (such as but not limited to having at least two PIR-based motion detection sensors of substantially similar maximum detection distances). In any case, disinfection apparatus 70 may, in some embodiments, include one or more additional motion detection sensors which do not have the configuration of a PIR-based motion detection multiple sensor coalesced beam pattern systems, including those which are not PIR based.

In yet other embodiments, disinfection apparatus 70 may have a PIR-based motion detection system of an entirely different configuration than as shown in FIG. 14, including but not limited to having individual PIR-based motion detection sensors individually arranged at respectively different locations of the apparatus such as described below in reference to disinfection apparatus 80 shown in FIG. 15. In particular, disinfection apparatus is not limited to having PIR-based motion detection multiple sensor coalesced beam pattern systems. Similarly, disinfection apparatus 80 may have a motion detection system of an entirely different configuration than as shown in FIG. 15, including but not limited to having PIR-based motion detection multiple sensor coalesced beam pattern systems such as described in reference to disinfection apparatus 70 shown in FIG. 14. As such, the type of motion detection system employed by disinfection apparatuses 70 and 80 are not mutually exclusive to the configuration of the respective disinfection apparatuses.

As shown in FIG. 15, PIR-based motion detection sensors 82, 83 and 84 are individually arranged at respectively different locations of disinfection apparatus 80, particularly spaced apart such that each sensor is disposed on its own printed circuit board. Together, PIR-based motion detection sensors 82, 83 and 84 may completely or partially make up a motion detection system for disinfection apparatus 80. PIR-based motion detection sensor 83 is shown as a different size than PIR-based motion detection sensors 82 and 84 to indicate it has a different detection range than PIR-based motion detection sensors 82 and 84. In alternative embodiments, PIR-based motion detection sensor 82 may have a different detection range than PIR-based motion detection sensors 83 and 84 or PIR-based motion detection sensor 84 may have a different detection range than PIR-based motion detection sensors 82 and 83. In yet other cases, each of PIR-based motion detection sensors 82-84 may have a different detection range. In any of such cases, the sequence of PIR-based motion detection sensors may continue around cap assembly 89, particularly when the PIR-based motion detection sensors are arranged to collectively monitor an area approximately 360° of motion detection around the disinfection apparatus.

In some cases, it may be advantage to have the PIR-based motion detection sensor with a different detection range disposed between two sensors of the same detection range, such as shown for the configuration in FIG. 15. In particular, an alternating sequence of PIR-based motion detection sensors having different detection ranges may be advantageous to make the area of motion detection adjacent (and sometimes around) the apparatus relatively uniform (at least to the maximum distance governed by the PIR-based motion detection sensor/s having the smaller detection distances). An alternating sequence of PIR-based motion detection sensors having different detection ranges may be particularly beneficial when the detection angles, detection ranges, and spacing of the PIR-based motion detection sensors with longer detection ranges are sufficient to merge their fields of views at a distance within their detection ranges. In particular, such a configuration allows motion to be detected at a relatively far distance from motion detection system 20 in a given area while also having relatively high sensitivity of detecting motion in a closer region in the same given area by the PIR-based motion detection sensors with shorter detection ranges. Furthermore, an alternating sequence of PIR-based motion detection sensors having different detection ranges may be advantageous for lessening or even eliminating the need for precisely orienting a disinfection device in a room such that the motion detection sensors having the larger detection range are oriented to monitor an area in a larger dimension of the room versus the motion detection sensors having the smaller detection range being oriented to monitor an area in a smaller dimension of the room.

In yet other cases, PIR-based motion detection sensors of different sizes may not be arranged in an alternating sequence on an apparatus. For instance, it may be advantageous to arrange PIR-based motion detection sensors having different detection ranges on respectively different sides (and in some cases opposing sides) of a mobile apparatus. In particular, such an arrangement may be beneficial to lessen the accuracy needed to position the mobile apparatus such that the motion detection sensors having the larger detection range are oriented to monitor an area in a larger dimension of the room versus the motion detection sensors having the smaller detection range being oriented to monitor an area in a smaller dimension of the room.

Regardless of the arrangement of their PIR-based motion detection sensors, the configuration of their individual components (such as but not limited to the number, type, or placement of their germicidal sources), and their configuration as a whole, the disinfection apparatuses described herein include detection circuitry independently and operatively coupled to the PIR sensors of their PIR-based motion detection sensors. In the disinfection apparatuses discussed in reference to FIGS. 14 and 15, the detection circuitry coupled between their PIR-based motion detection sensors and their germicidal source/s may be respectively disposed within mobile carriages 78 and 88, but other configurations may be considered. In any case, the detection circuitry of the disinfection apparatuses considered herein is configured to send a signal to operational circuitry of the germicidal source/s of a respective apparatus upon receipt of a threshold signal from a PIR sensor on the apparatus that indicates a predetermined change of infrared radiation has been detected at the respective PIR as similarly described above for the detection circuitry discussed in reference to FIG. 13. In addition, the operational circuitry of the germicidal source/s in the disinfection apparatuses discussed herein are configured to affect operation of the germicidal source/s subsequent to receipt of the signal from the detection circuitry.

The signal sent by the detection circuitry may be a signal to inhibit operation of the germicidal source/s or may be a signal to terminate operation of the germicidal source/s. In particular, the PIR-based motion detection sensors of the disinfection apparatuses described herein may be used to monitor for movement in an area/room before the apparatus starts a disinfection cycle and/or may be used to monitor for movement while the apparatus is conducting a disinfection cycle. Accordingly, in cases in which PIR-based motion detection sensors are used to monitor for movement in a room before an apparatus starts a disinfection cycle, the signal sent by its detection circuitry may be a signal to inhibit operation of its germicidal source/s (herein referred to as an “inhibitory signal”). In other cases, in embodiments in which PIR-based motion detection sensors are used to monitor for movement in a room while an apparatus is conducting a disinfection cycle, the signal sent by the detection circuitry may be a signal to terminate operation of its germicidal source/s (herein referred to as a “termination signal”).

Although the disinfection apparatuses disclosed herein may have distinct sets of PIR-based motion detection sensors for respectively monitoring motion in a room before and during a disinfection cycle, in some configurations a set of PIR-based motion detection sensors may be used for detecting motion in both stages of an apparatus being used to disinfect an area/room. In some of such cases, the detection circuitry may be configured to detect whether the apparatus's germicidal source/s are in operation and accordingly send either an inhibitory signal or a termination signal to the germicidal source/s. In other cases, the signal from the detection circuitry may not be dependent on detecting whether the germicidal source/s are in operation and may not be specific to inhibiting or terminating operation of the germicidal source/s. Instead, the detection circuitry may send a general signal to affect operation of the germicidal source/s and the germicidal source/s themselves may be configured to detect its current operational state and accordingly inhibit its operation or terminate its operation upon receipt of a signal from the detection circuitry.

As noted above, FIG. 16 illustrates an example top view of an apparatus having multiple PIR sensors arranged thereon to collectively provide 360° of motion detection around the apparatus. In particular, FIG. 16 illustrates an example arrangement of PIR-based motion detection sensors 92 along sidewalls of apparatus 90 as taken from a top view of the apparatus, specifically having two sensors along opposing sides of the apparatus and another two sensors respectively at other opposing sides (e.g., the front side and the back side) of the apparatus. The arrangement of PIR-based motion detection sensors 92 is specific to apparatus 90 being oblong, but other arrangements may be considered, depending on the detection ranges and detection angles of the motion detection sensors. Furthermore, apparatuses having other circumferential shapes may be considered for arranging multiple PIR sensors thereon to collectively provide 360° of motion detection around the apparatus. In any case, PIR-based motion detection sensors 92 may take any form described herein for PIR-based motion detection sensors, specifically as PIR-based motion detection multiple sensor coalesced beam pattern systems with PIR-based motion detection sensors having different or the same detection ranges or as individual PIR-based motion detection sensors having different detection ranges but which are not vertically disposed and/or have central axes of their optical systems substantially aligned.

As shown in FIG. 16, the arrangement of PIR-based motion detection sensors 92 along the sides of apparatus 90 as well as their detection ranges and detection angles are such that the fields of view of the sensors overlap. Such a configuration collectively enables the ability to detect motion approximately 360° around the apparatus as shown in FIG. 16. In some cases, the positions, detection ranges, and detection angles of PIR-based motion detection sensors 92 are such that the area of unmonitored regions 94 are minimized, particularly such the maximum distance of each of unmonitored regions 94 is less than 1 foot from apparatus 90 and, in some cases, less than 6 inches from apparatus 90. In particular, such a minimization of unmonitored regions 94 may be beneficial for ensuring motion in close proximity to apparatus 90 may be detected.

It may be particularly advantageous to be able to detect motion 360° around a disinfection apparatus in regions both close and far from the apparatus when PIR-based motion detection sensors 92 are to be used to monitor for movement in an area/room before the disinfection apparatus starts a disinfection cycle. In particular, some disinfection apparatuses may be configured to activate a disinfection cycle in response to input on a user interface on the apparatus and further configured to delay activation of its germicidal source for a predetermined amount of time to ensure the area/room has been vacated before the germicidal source is activated. In such cases, it would be advantageous to be able to detect motion 360° around a disinfection apparatus in regions close and far from the apparatus to ensure a user of the apparatus has left the vicinity of the apparatus and has exited the area/room. Such a scenario differs from a scenario in which PIR-based motion detection sensors 92 are just to be used to monitor for movement in an area/room during a disinfection cycle since the area/room is presumably vacated prior to activating its germicidal source and, thus, any movement occurring during a disinfection cycle will generally be near entryways into the area/room, which depending on the position of the disinfection apparatus in the room will generally be at least 2 feet from the disinfection apparatus.

Although PIR-based motion detection sensors with wider detection angles and/or additional PIR-based motion detection sensors along the sides of apparatus may further reduce the size of unmonitored regions 94, such variations may be disadvantageous in some cases. In particular, adding sensors generally increases the complexity of the detection circuity of the motion detection system and increases the cost of manufacturing the apparatus. In addition, PIR-based motion detection sensors with wider detection angles generally have lower detection efficacy. However, such a disadvantage may be mitigated if one or more of PIR-based motion detection sensors 92 are PIR-based motion detection multiple sensor coalesced beam pattern systems.

It will be appreciated to those skilled in the art having the benefit of this disclosure that this invention is believed to provide motion detection systems having at least two passive infrared motion detection sensors arranged such that their respective beam patterns increase detection sensitivity of the system. Further modifications and alternative embodiments of various aspects of the invention will be apparent to those skilled in the art in view of this description. For example, although the discussion provided herein emphasizes use of such motion detection systems with an area/room disinfection apparatus, the motion detection systems may be used with other apparatuses or in conjunction with other systems. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims. The term “approximately” as used herein refers to variations of up to +/−5% of the stated number. The term “substantially similar” as used herein refers to variations of up to +/−5% between two values of a stated parameter.

What is claimed is:

1. A motion detection system, comprising:
a first motion detection sensor comprising a first passive infrared sensor (first PIR) and a first Fresnel lens array for focusing infrared radiation onto the first PIR;
a second motion detection sensor comprising a second passive infrared sensor (second PIR) and a second Fresnel lens array for focusing infrared radiation onto the second PIR, wherein the second Fresnel lens array comprises a different configuration of lenses than the first Fresnel lens array;
a substrate to which the first motion detection sensor and the second motion detection sensor are attached, wherein the first and second motion detection sensors are arranged on the substrate such that sensing elements of the first PIR and sensing elements of the second PIR can detect motion along the same plane in an ambient of the motion detection system, and wherein when the substrate is arranged such that the sensing elements of the first and second PIRs are oriented to detect motion along a horizontal plane in the ambient of the motion detection system:
the first motion detection sensor is arranged above and over the second motion detection sensor; and
the first and second Fresnel lens arrays are configured such that a composite overlay of their horizontal fields of views has a beam pattern associated with the first Fresnel lens array overlapping a majority portion of dead zones associated with the second Fresnel lens array; and
detection circuitry independently and operatively coupled to the first PIR and the second PIR, wherein the detection circuitry is configured to produce a motion detection signal upon receipt of a threshold signal from either the first PIR or the second PIR that indicates a predetermined change of infrared radiation has been detected at the respective PIR.

2. The motion detection system of claim 1, wherein the beam pattern of the associated with the first Fresnel lens array is non-uniform.

3. The motion detection system of claim 1, wherein the composite overlay has a beam pattern associated with the second Fresnel lens array underlying a majority portion of dead zones associated with the first Fresnel lens array.

4. The motion detection system of claim 1, wherein at least a portion of the composite overlay comprises a sequence of beams which alternate between the beam pattern associated with the first Fresnel lens array and the beam pattern associated with the second Fresnel lens array.

5. The motion detection system of claim 1, wherein when the first motion detection sensor is arranged above and over the second motion detection sensor the horizontal field of view of the first Fresnel lens array or the second Fresnel lens array comprises a dead zone along and extending from its central axis.

6. The motion detection system of claim 1, wherein when the first motion detection sensor is arranged above and over the second motion detection sensor the first motion detection sensor is oriented relative to the second motion detection sensor such that central axes of the first and second Fresnel lens arrays are substantially aligned as viewed from a top view of the motion detection system.

7. The motion detection system of claim 1, wherein when the first motion detection sensor is arranged above and over the second motion detection sensor the first motion detection sensor is oriented relative to the second motion detection sensor such that central axes of the first and second Fresnel lens arrays are substantially parallel as viewed from a side view of the motion detection system.

8. A motion detection system, comprising:
a first motion detection sensor comprising a first passive infrared sensor (first PIR) and a first optical system for focusing infrared radiation onto the first PIR, wherein the first optical system is configured to receive infrared radiation emanating from a first plurality of detection zones in an ambient of the first motion detection sensor;
a second motion detection sensor comprising a second passive infrared sensor (second PIR) and a second optical system for focusing infrared radiation onto the second PIR, wherein the second optical system is configured to receive infrared radiation emanating from a second plurality of detection zones in an ambient of the second motion detection sensor, wherein one of the first and second motion detection sensors is vertically disposed above the other of the first and second motion detection sensors, and wherein each of the first and second optical systems have a horizontal field of view embodying:
a first preset area extending from a central axis of their optical system to a same first preset angle relative to the central axis, wherein the first plurality of detection zones comprises a higher density of detection zones in its respective first preset area than the second plurality of detection zones comprises in its respective first preset area; and
a second preset area extending from the same first preset angle to a same second preset angle relative to their central axis, wherein the second plurality of detection zones comprises a higher density of detection zones in its respective second preset area than the first plurality of detection zones comprises in its respective second preset area; and
detection circuitry independently and operatively coupled to the first PIR and the second PIR, wherein the detection circuitry is configured to produce a motion detection signal upon receipt of a threshold signal from either the first PIR or the second PIR that indicates a predetermined change of infrared radiation has been detected at the respective PIR.

9. The motion detection system of claim 8, wherein the first preset area of each of the horizontal fields of view of the first and second optical systems further extends to a same third preset angle on an opposing side of their respective central axis from which the first preset angle extends.

10. The motion detection system of claim 8, wherein the horizontal field of view of each of the first and second optical systems embodies a third preset area extending from the same second preset angle to their respective maximum detection angle.

11. The motion detection system of claim 8, wherein the first PIR and the second PIR each comprise sensing elements oriented to detect horizontal movement in an ambient of the motion detection system.

12. The motion detection system of claim 8, wherein the second motion detection sensor is oriented relative to the first motion detection sensor such that the central axes of the first and second optical systems are substantially aligned.

13. The motion detection system of claim 8, wherein the second motion detection sensor is oriented relative to the first motion detection sensor such that the central axes of the first and second optical systems are substantially parallel.

14. The motion detection system of claim 8, wherein the first and second optical systems each comprise a Fresnel lens array and/or segmented mirrors.

15. An apparatus, comprising:
a germicidal source, wherein the apparatus is configured such that a germicide emitted from the germicidal source is projected exterior to the apparatus; and
a motion detection system comprising:
a first motion detection sensor comprising a first passive infrared sensor (first PIR) and a first optical system for focusing infrared radiation onto the first PIR;
a second motion detection sensor comprising a second passive infrared sensor (second PIR) and a second optical system for focusing infrared radiation onto the second PIR, wherein the first and second motion detection sensors comprise different maximum detection distances; and
detection circuitry independently and operatively coupled to the first PIR and the second PIR, wherein the detection circuitry is configured to send a motion detection signal to operational circuitry of the germicidal source upon receipt of a threshold signal from either the first PIR or the second PIR that indicates a predetermined change of infrared radiation has been detected at the respective PIR, and wherein the operational circuitry is configured to affect operation of the germicidal source subsequent to receipt of the motion detection signal.

16. The apparatus of claim 15, wherein each of the different detection distances are greater than approximately 6 meters.

17. The apparatus of claims 15, wherein the first motion detection sensor is arranged above and over the second motion detection sensor.

18. The apparatus of claim 15, wherein the first and second motion detection sensors are arranged such that central axes of the first and second optical systems are substantially aligned.

19. The apparatus of claim 15, wherein the first and second motion detection sensors are arranged such that central axes of the first and second optical systems are substantially parallel.

20. The apparatus of claim 15, wherein the apparatus is configured is to project an effective amount of germicide into an ambient of the apparatus to achieve at least a 2-log reduction in bacterial contamination on surfaces that are greater than 1 meter from the germicidal source.
